SEQUENCE LISTING (42 sequences)

SEQ ID NO: 1 - protein, 10 aa, synthetic construct
  VARIANT 10: note = Y, W, or F
LSSTQAQQSX

SEQ ID NO: 2 - protein, 10 aa, synthetic construct
LSSTQAQQSY

SEQ ID NO: 3 - protein, 10 aa, synthetic construct
LSSTQAQQSW

SEQ ID NO: 4 - protein, 10 aa, synthetic construct
LSSTQAQQSF

SEQ ID NO: 5 - protein, 5 aa, synthetic construct
SSTQA

SEQ ID NO: 6 - protein, 5 aa, synthetic construct
STQAQ

SEQ ID NO: 7 - protein, 5 aa, synthetic construct
TQAQQ

SEQ ID NO: 8 - protein, 5 aa, synthetic construct
QAQQS

SEQ ID NO: 9 - protein, 5 aa, synthetic construct
AQQSW

SEQ ID NO: 10 - protein, 5 aa, synthetic construct
AQQSF

SEQ ID NO: 11 - protein, 5 aa, synthetic construct
LSSTQ

SEQ ID NO: 12 - protein, 5 aa, synthetic construct
AQQSY

SEQ ID NO: 13 - protein, 6 aa, synthetic construct
LSSTQA

SEQ ID NO: 14 - protein, 6 aa, synthetic construct
SSTQAQ

SEQ ID NO: 15 - protein, 6 aa, synthetic construct
STQAQQ

SEQ ID NO: 16 - protein, 6 aa, synthetic construct
TQAQQS

SEQ ID NO: 17 - protein, 6 aa, synthetic construct
QAQQSY

SEQ ID NO: 18 - protein, 6 aa, synthetic construct
QAQQSW

SEQ ID NO: 19 - protein, 6 aa, synthetic construct
QAQQSF

SEQ ID NO: 20 - protein, 6 aa, synthetic construct
WLSSTQ

SEQ ID NO: 21 - protein, 6 aa, synthetic construct
AQQSYW

SEQ ID NO: 22 - protein, 7 aa, synthetic construct
LSSTQAQ

SEQ ID NO: 23 - protein, 7 aa, synthetic construct
SSTQAQQ

SEQ ID NO: 24 - protein, 7 aa, synthetic construct
STQAQQS

SEQ ID NO: 25 - protein, 7 aa, synthetic construct
TQAQQSY

SEQ ID NO: 26 - protein, 7 aa, synthetic construct
TQAQQSW

SEQ ID NO: 27 - protein, 7 aa, synthetic construct
TQAQQSF

SEQ ID NO: 28 - protein, 8 aa, synthetic construct
LSSTQAQQ

SEQ ID NO: 29 - protein, 8 aa, synthetic construct
SSTQAQQS

SEQ ID NO: 30 - protein, 8 aa, synthetic construct
STQAQQSY

SEQ ID NO: 31 - protein, 8 aa, synthetic construct
STQAQQSW

SEQ ID NO: 32 - protein, 8 aa, synthetic construct
STQAQQSF

SEQ ID NO: 33 - protein, 9 aa, synthetic construct
SSTQAQQSW

SEQ ID NO: 34 - protein, 9 aa, synthetic construct
SSTQAQQSF

SEQ ID NO: 35 - protein, 9 aa, synthetic construct
LSSTQAQQS

SEQ ID NO: 36 - protein, 9 aa, synthetic construct
SSTQAQQSY

SEQ ID NO: 37 - skipped

SEQ ID NO: 38 - protein, 11 aa, synthetic construct
LSSTQAQQSYW

SEQ ID NO: 39 - protein, 11 aa, synthetic construct
WLSSTQAQQSY

SEQ ID NO: 40 - protein, 12 aa, synthetic construct
WLSSTQAQQSYW

SEQ ID NO: 41 - protein, 14 aa, synthetic construct
ESFFLSSTQAQQSY

SEQ ID NO: 42 - protein, 30 aa, synthetic construct
  VARIANT 20: note = Y, W, or F
NKPGRFESFFLSSTQAQQSXLQGFSKNILE

(19) United States
(12) Patent Application Publication
(10) Pub. No.: US 2025/0059233 A1
(43) Pub. Date: Feb. 20, 2025

(54) USE OF THERAPEUTIC PEPTIDES

(71) Applicants: Viage Therapeutics Inc., Palo Alto, CA (US); Kyoto University, Kyoto (JP)

(72) Inventors: Igor Grachev, Millstone Twp, NJ (US); Sooin Hwang, San Rafael, CA (US); Takahide Koda, Yokohama (JP); Harald Murk, Westfield, NJ (US); Takashi Nishida, Kyoto-shi (JP); Kousaku Ohinata, Kyoto-shi (JP)

(21) Appl. No.: 18/720,588

(22) PCT Filed: Dec. 14, 2022
(86) PCT No.: PCT/US2022/081558

§ 371 Date: Jun. 14, 2024

Related U.S. Application Data
(60) Provisional application No. 63/289,915, filed on Dec. 15, 2021.

(51) Int. Cl. C07K 7/06

(52) U.S. Cl. CPC C07K 7/06

(57) ABSTRACT

Peptides and pharmaceutically acceptable salts thereof and peptide conjugates and pharmaceutically acceptable salts thereof having 5 to 15 amino acids and having an amino acid sequence comprising at least 5 consecutive amino acids from the amino acid sequence LSSTQAQQSX₁ (SEQ ID NO:1) for use in treating a subject suffering from or at risk of an inflammatory disease or disorder, schizophrenia or psychosis, a neurodegenerative disease such as Parkinson's disease. Alzheimer's disease, or ALS, a gastrointestinal disease or disorder such as irritable bowel syndrome, inflammatory bowel disease. Crohn's disease, or constipation, pain (e.g., visceral pain), rheumatoid arthritis, migraine, headache, substance abuse (e.g., substance use disorder such as drug use disorder, polysubstance use disorder, alcohol use disorder, or nicotine use disorder, tobacco use disorder), drug addiction, a seizure disorder, major depressive disorder, atypical depression, a major depressive episode (MDE) (e.g., atypical MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder, or for use in improving sleep in a subject.

### 1. CROSS-REFERENCE TO RELATED APPLICATIONS

This application claims the priority benefit of U.S. provisional application No. 63/289,915, filed Dec. 15, 2021, the contents of which are incorporated herein in their entireties by reference thereto.

### 2. SEQUENCE LISTING

The instant application contains a Sequence Listing XML which has been submitted electronically and is hereby incorporated by reference in its entirety. Said Sequence Listing XML, created on Dec. 9, 2022, is named DGS-005WO_SL.xml and is 37.105 bytes in size.

### 3. BACKGROUND

PCT international publication nos. WO 2016/140277 and WO 2018/047852 describe peptides and peptide conjugates, for example, LSSTQAQQSY (SEQ ID NO:2), useful for treating mental disorders such as mood disorders, anxiety disorders, and disorders of diminished motivation.

### 4. SUMMARY

The present disclosure is based, in part, on new insights into the biological activities of the peptides and peptide conjugates described in WO 2016/140277 and WO 2018/047852. Specifically, it has been surprisingly discovered that the peptide LSSTQAQQSY (SEQ ID NO:2) has biological activities beyond those previously reported. For example, microdialysis studies performed with the peptide LSSTQAQQSY (SEQ ID NO:2), detailed in Section 7, demonstrated that oral administration of the peptide surprisingly affects the levels of certain neurotransmitters in the brain, including histamine and norepinephrine. Without being bound by theory, these neurotransmitters are believed to be associated with various diseases, disorders, and conditions, for example schizophrenia and neurodegenerative diseases (e.g., Parkinson's disease), and in particular negative symptoms associated with these diseases. Further, a study performed with the peptide LSSTQAQQSY (SEQ ID NO:2) in a lipopolysaccharide-induced model of inflammation, also detailed in Section 7, demonstrated that oral administration of the peptide surprisingly suppresses release of TNF-α, an inflammatory cytokine associated with various diseases, including rheumatoid arthritis and inflammatory bowel disease. Further still, a study performed with the peptide LSSTQAQQSY (SEQ ID NO:2) in a scopolamine-induced model of memory impairment, also detailed in Section 7, demonstrated that oral administration of the peptide surprisingly led to an improvement in memory. Further still, a study performed with the peptide LSSTQAQQSY (SEQ ID NO:2) in human subjects, also detailed in Section 7, demonstrated that oral administration of the peptide provides pro-cognitive effects. Thus, the studies described in Section 7 indicate that the peptides and peptide conjugates described herein can be used to treat diseases, disorders, and conditions beyond those described in WO 2016/140277 and WO 2018/047852.

Accordingly, in some aspects, the disclosure provides methods for treating diseases, disorders and conditions amenable to treatment by vagus nerve stimulation and/or modulation of histamine and/or norepinephrine levels and/or modulation of TNF-α. In various aspects, the disclosure provides methods of treating subjects suffering from or at risk of an inflammatory disease or condition, schizophrenia or psychosis, a neurodegenerative disease such as Parkinson's disease. Alzheimer's disease, or amyotrophic lateral sclerosis (ALS), a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction (e.g., opioid, for example morphine, heroin, oxycodone, or fentanyl; cocaine; or benzodiazepine such as diazepam, alprazolam, or clonazepam), a seizure disorder, major depressive disorder, atypical depression, major depressive episode (MDE) (e.g., atypical MDE), depression in the presence of a neurodegenerative disease (e.g., Parkinson's disease, Alzheimer's disease, or ALS), cognitive impairment, COVID-19 related cognitive impairment and/or depression, ADHD, an autism spectrum disorder, a pervasive developmental disorder, pervasive developmental disorders not otherwise specified (also called atypical autism), multiple sclerosis, post-traumatic stress disorder (PTSD), a sleep disorder such as insomnia, daytime fatigue, or REM sleep behavior disorder, and methods of improving sleep, the methods comprising administering a peptide or salt thereof, peptide conjugate or salt thereof, or pharmaceutical composition described herein (collectively referred to herein as “compounds and compositions of the disclosure” for convenience) to a subject in need thereof. In some embodiments, the subject has an elevated TNF-α level.

In further aspects, the disclosure provides the compounds and compositions of the disclosure for use in a method of treatment described herein.

In further aspects, the disclosure provides uses of the compounds and composition of the disclosure in the manufacture of medicaments for treating subjects having or at risk of developing a disease or condition disclosed herein.

Exemplary features of methods of treatment, compounds and compositions for use in a method of treatment, and uses are described in Section 6.4 and numbered embodiments 1 to 461, infra.

### 5. BRIEF DESCRIPTION OF THE FIGURES

FIGS. 1A-1G show neurotransmitter levels in mouse prefrontal cortex before and after oral administration of LSSTQAQQSY (SEQ ID NO:2) (Example 1). FIG. 1A: histamine; FIG. 1B: norepinephrine; FIG. 1C: GABA; FIG. 1D: glutamate; FIG. 1E: glycine; FIG. 1F: dopamine; FIG. 1G: serotonin.

FIGS. 2A-2G show neurotransmitter levels in mouse striatum before and after oral administration of LSSTQAQQSY (SEQ ID NO:2) (Example 1). FIG. 2A: histamine; FIG. 2B: norepinephrine; FIG. 2C: GABA; FIG. 2D: glutamate; FIG. 2E: glycine; FIG. 2F: dopamine; FIG. 2G: serotonin.

FIG. 3 shows plasma TNF-α levels following administration of lipopolysaccharide and the peptide LSSTQAQQSY (SEQ ID NO:2) (Example 2).

FIG. 4 shows latency of step-down inhibitor avoidance test in mice (n=10, Mean±SEM) (Example 5). **P<0.01 vs Model group, *P<0.05 vs Model group (t-test for the paired comparisons).

FIG. 5 shows that the AVPR1A/AVPR2 inhibitor conivaptan blocks LSSTQAQQSY (SEQ ID NO:2) activity in a tail suspension test (Example 6).

FIG. 6 shows that low doses of the AVPR1A/AVPR2 inhibitor conivaptan blocks LSSTQAQQSY (SEQ ID NO:2) activity in a tail suspension test (Example 6).

FIG. 7 shows that the AVPR2 inhibitor tolvaptan does not block LSSTQAQQSY (SEQ ID NO: 2) activity in a tail suspension test (Example 6).

FIG. 8 shows that the AVPR1A/B inhibitor nelivaptan blocks LSSTQAQQSY (SEQ ID NO: 2) activity in a tail suspension test (Example 6).

FIG. 9 shows the effect of doses of 60 mg/day (MAD1), 180 mg/day (MAD2), or 540 mg/day (MAD3), versus placebo, on overall results of the PHQ-9 questionnaire (Example 7).

FIG. 10 shows the effect of doses of 60 mg/day (MAD1), 180 mg/day (MAD2), or 540 mg/day (MAD3), versus placebo, on the CogState Detection Test (DET) of psychomotor function (Example 7).

FIG. 11 shows the effect of doses of 60 mg/day (MAD1), 180 mg/day (MAD2), or 540 mg/day (MAD3), versus placebo, on the CogState Groton Maze Learning Test (GMLT) of executive function (Example 7).

FIG. 12 shows the effect of doses of 60 mg/day (MAD1), 180 mg/day (MAD2), or 540 mg/day (MAD3), versus placebo, on the CogState Identification Test (IDN) of attention (Example 7).

### 6. DETAILED DESCRIPTION

### 6.1. Definitions

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). In certain embodiments, all amino acid substitutions as compared to SEQ ID NO:42 are conservative.

As used herein, the term “negative symptom” refers to a deficit in the ability of a subject to perform a normal function. Examples of negative symptoms, for example in a subject suffering from schizophrenia, include asociality, anhedonia, alogia, affective flattening, apathy, avolition, blunted affect, anergia, depression, low mood, and cognitive impairment. Negative symptoms, e.g., one or more of the foregoing negative symptoms, can also be experienced by subjects suffering from other diseases, for example subjects having a neurodegenerative disease, for example Parkinson's disease, Alzheimer's disease, or ALS. Common negative symptoms in such subjects include anhedonia, apathy, depression, and cognitive impairment.

As used herein, a “non-motor symptom” of Parkinson's disease refers to a symptom that is not related to movement. Non-motor symptoms include, for example, cognitive symptoms such as mood changes, depression, anxiety, panic attacks, tiredness, confusion, and slowed thinking, sensory symptoms such as numbness, restlessness, pain, chest discomfort, and anosmia, and autonomic symptoms such as hot/cold sensations, bladder problems, sweating, abdominal discomfort, constipation, sialorrhea, frequent urination and/or urgency, and erectile dysfunction.

As used herein, the terms “treat”. “treatment” and “treating” refer to the reduction or amelioration of the progression or severity of one or more symptoms (preferably, one or more discernible symptoms) of a disease, disorder, or condition resulting from the administration of a compound or composition of the disclosure. In specific embodiments, the terms “treat”, “treatment” and “treating” refer to the amelioration of at least one measurable physical parameter of a disease, disorder, or condition. In other embodiments, the terms “treat”, “treatment” and “treating” refer to the slowing of the progression of one or more symptoms of a disease, disorder, or condition.

### 6.2. Peptides and Peptide Conjugates

Peptides that can be used in the compositions and methods of the disclosure are 5 to 15 amino acids in length and have an amino acid sequence comprising at least 5 consecutive amino acids from the amino acid sequence LSSTQAQQSX₁ (SEQ ID NO:1), where X₁ is Y, W, of F. In certain embodiments, the peptides are 5-8, 6-10, 7-11, 8-12, 9-13, 10-14, 11-15 or 12-15 amino acids in length. Exemplary peptides and salts thereof are described in Section 6.2.1.

Peptide conjugates and salts thereof can also be used in the compositions and methods of the disclosure. The peptide conjugates comprise a peptide moiety attached to one or more (e.g., one, two or three) conjugate moieties. The peptide moiety can be 5 to 15 amino acids in length and comprise at least 5 consecutive amino acids from the amino acid sequence LSSTQAQQSX₁ (SEQ ID NO:1), where X₁ is Y, W, of F. In certain embodiments, the peptide moieties are 5-8, 6-10, 7-11, 8-12, 9-13, 10-14, 11-15 or 12-15 amino acids in length. Exemplary peptide conjugates and salts thereof are described in Section 6.2.2.

### 6.2.1. Peptides

Peptides and salts thereof that can be used in the compositions and methods of the disclosure have an amino acid sequence that comprises or consists of at least 5 consecutive amino acids from the amino acid sequence LSSTQAQQSX₁ (SEQ ID NO:1), where X₁ is Y, W, or F. It should be understood that when an embodiment described herein refers to a “peptide,” the embodiment encompasses the peptide per se as well as salts of the peptide even though the embodiment may not explicitly recite the expression “or salt thereof” or similar, unless required otherwise by context.

The peptides can be 5 to 15 amino acids (i.e., 5, 6, 7, 8, 9, 10, 11, 12, 13, 14 or 15 amino acids) in length. In some embodiments, a peptide is 5 amino acids in length. In other embodiments, a peptide is 6 amino acids in length. In other embodiments, a peptide is 7 amino acids in length. In other embodiments, a peptide is 8 amino acids in length. In other embodiments, a peptide is 9 amino acids in length. In other embodiments, a peptide is 10 amino acids in length. In other embodiments, a peptide is 11 amino acids in length. In other embodiments, a peptide is 12 amino acids in length. In other embodiments, a peptide is 13 amino acids in length. In other embodiments, a peptide is 14 amino acids in length. In other embodiments, a peptide is 15 amino acids in length.

The peptides generally comprise or consist of at least 5 consecutive amino acids from the amino acid sequence LSSTQAQQSX₁ (SEQ ID NO:1). Accordingly, in some embodiments, the peptide comprises or consists of the amino acid sequence SSTQA (SEQ ID NO:5). In other embodiments, the peptide comprises or consists of the amino acid sequence STQAQ (SEQ ID NO:6). In other embodiments, the peptide comprises or consists of the amino acid sequence TQAQQ (SEQ ID NO:7). In other embodiments, the peptide comprises or consists of the amino acid sequence QAQQS (SEQ ID NO:8). In other embodiments, the peptide comprises or consists of the amino acid sequence AQQSW (SEQ ID NO:9). In other embodiments, the peptide comprises or consists of the amino acid sequence or AQQSF (SEQ ID NO:10). In other embodiments, the peptide comprises or consists of the amino acid sequence LSSTQ (SEQ ID NO:11). In other embodiments, the peptide comprises or consists of the amino acid sequence AQQSY (SEQ ID NO:12).

In certain aspects, a peptide comprises or consists of at least 6 consecutive amino acids from the amino acid sequence LSSTQAQQSX₁ (SEQ ID NO:1). Accordingly, in some embodiments, the peptide comprises or consists of the amino acid sequence LSSTQA (SEQ ID NO:13). In other embodiments, the peptide comprises or consists of the amino acid sequence SSTQAQ (SEQ ID NO:14). In other embodiments, the peptide comprises or consists of the amino acid sequence STQAQQ (SEQ ID NO:15). In other embodiments, the peptide comprises or consists of the amino acid sequence TQAQQS (SEQ ID NO:16). In other embodiments, the peptide comprises or consists of the amino acid sequence QAQQSY (SEQ ID NO:17). In other embodiments, the peptide comprises or consists of the amino acid sequence QAQQSW (SEQ ID NO:18). In other embodiments, the peptide comprises or consists of the amino acid sequence QAQQSF (SEQ ID NO:19). In other embodiments, the peptide comprises or consists of the amino acid sequence WLSSTQ (SEQ ID NO:20). In other embodiments, the peptide comprises or consists of the amino acid sequence AQQSYW (SEQ ID NO:21).

In other aspects, a peptide comprises or consists of at least 7 consecutive amino acids from the amino acid sequence LSSTQAQQSX₁ (SEQ ID NO:1). Accordingly, in some embodiments, the peptide comprises or consists of the amino acid sequence LSSTQAQ (SEQ ID NO:22). In other embodiments, the peptide comprises or consists of the amino acid sequence SSTQAQQ (SEQ ID NO:23). In other embodiments, the peptide comprises or consists of the amino acid sequence STQAQQS (SEQ ID NO:24). In other embodiments, the peptide comprises or consists of the amino acid sequence TQAQQSY (SEQ ID NO:25). In other embodiments, the peptide comprises or consists of the amino acid sequence TQAQQSW (SEQ ID NO:26). In other embodiments, the peptide comprises or consists of the amino acid sequence TQAQQSF (SEQ ID NO:27).

In yet other aspects, a peptide comprises or consists of at least 8 consecutive amino acids from the amino acid sequence LSSTQAQQSX₁ (SEQ ID NO:1). Accordingly, in some embodiments, the peptide comprises or consists of the amino acid sequence LSSTQAQQ (SEQ ID NO:28). In other embodiments, the peptide comprises or consists of the amino acid sequence SSTQAQQS (SEQ ID NO:29). In other embodiments, the peptide comprises or consists of the amino acid sequence STQAQQSY (SEQ ID NO:30). In other embodiments, the peptide comprises or consists of the amino acid sequence STQAQQSW (SEQ ID NO:31). In other embodiments, the peptide comprises or consists of the amino acid sequence STQAQQSF (SEQ ID NO:32).

In yet further aspects, a peptide comprises or consists of at least 9 consecutive amino acids from the amino acid sequence LSSTQAQQSX₁ (SEQ ID NO:1). Accordingly, in some embodiments, the peptide comprises or consists of the amino acid sequence SSTQAQQSW (SEQ ID NO:33). In other embodiments, the peptide comprises or consists of the amino acid sequence SSTQAQQSF (SEQ ID NO:34). In other embodiments, the peptide comprises or consists of the amino acid sequence LSSTQAQQS (SEQ ID NO:35). In other embodiments, the peptide comprises or consists of the amino acid sequence SSTQAQQSY (SEQ ID NO:36).

In yet further aspects, a peptide comprises or consists of 10 consecutive amino acids from the amino acid sequence LSSTQAQQSX; (SEQ ID NO:1) and is 10, 11, 12, 13, 14, or 15 amino acids in length. In some embodiments, the peptide comprises or consists of the amino acid sequence LSSTQAQQSY (SEQ ID NO:2). In other embodiments, the peptide comprises or consists of the amino acid sequence LSSTQAQQSW (SEQ ID NO:3). In other embodiments, the peptide comprises or consists of the amino acid sequence LSSTQAQQSF (SEQ ID NO:4).

In other embodiments, the peptide comprises or consists of LSSTQAQQSYW (SEQ ID NO:38). In other embodiments, the peptide comprises or consists of WLSSTQAQQSY (SEQ ID NO:39). In other embodiments, the peptide comprises or consists of WLSSTQAQQSYW (SEQ ID NO:40). In other embodiments, the peptide comprises or consists of ESFFLSSTQAQQSY (SEQ ID NO:41).

Peptides that are 6 to 15 amino acids in lengths (i.e., peptides that are 6, 7, 8, 9, 10, 11, 12, 13, 14, or 15 amino acids long) can comprise, for example, an amino acid sequence identified in one of the previously described embodiments (i.e., comprise a sequence having at least 5, 6, 7, 8, 9, or 10 consecutive amino acids from SEQ ID NO:1) and one or more amino acids that naturally flank the sequence LSSTQAQQSY (SEQ ID NO:2) in the soybean storage protein β-conglycinin. For example, a peptide comprising the amino acid sequence LSSTQ (SEQ ID NO:11) and that is 6, 7, 8, 9, 10, 11, 12, 13, 14, or 15 amino acids in length can comprise an amino acid sequence N-terminal to the LSSTQ (SEQ ID NO:11) sequence corresponding to the 1, 2, 3, 4, 5, 6, 7, 8, 9, or 10 amino acids that are N-terminal to LSSTQAQQSY (SEQ ID NO:2) in the sequence of β-conglycinin. As another example, a peptide comprising the amino acid sequence AQQSY (SEQ ID NO:12) and that is 6, 7, 8, 9, 10, 11, 12, 13, 14, or 15 amino acids in length can comprise an amino acid sequence C-terminal to the AQQSY (SEQ ID NO:12) corresponding to 1, 2, 3, 4, 5, 6, 7, 8, 9, or 10 amino acids that are C-terminal to LSSTQAQQSY (SEQ ID NO:2) in the sequence of β-conglycinin.

NKPGRFESFFLSSTQAQQSX₁LQGFSKNILE (where X₁ is Y, W, of F) (SEQ ID NO:42) is a 30-mer peptide consisting of LSSTQAQQSX₁ (SEQ ID NO:1) flanked by 10 amino acids at each of the N- and C-termini that are present in the sequence of β-conglycinin. Thus, the first 10 amino acids in SEQ ID NO:42 represent the 10 amino acids N-terminal to LSSTQAQQSY (SEQ ID NO:2) in the sequence of β-conglycinin and the last 10 amino acids in SEQ ID NO:42 represent the 10 amino acids C-terminal to LSSTQAQQSY (SEQ ID NO:2) in the sequence of β-conglycinin.

In certain aspects, the sequence of a peptide used in the compositions and methods of the disclosure is derived from SEQ ID NO:42. Accordingly, the present disclosure provides a peptide which is 5 to 15 amino acids in length and comprises or consists of 5 to 15 consecutive amino acids from SEQ ID NO:42, provided that the peptide comprises at least 5 consecutive amino acids from the amino acid sequence LSSTQAQQSX: (SEQ ID NO:1).

Peptides having sequence variations relative to SEQ ID NO:42 are also contemplated herein. Accordingly, in other embodiments, the peptide can have an amino acid sequence comprising one or more amino acid substitutions as compared to an amino acid sequence consisting of 5 to 15 consecutive amino acids from SEQ ID NO:42, provided that the peptide comprises at least 5 consecutive amino acids from the amino acid sequence LSSTQAQQSX₁ (SEQ ID NO:1) that have not been substituted. The maximum number of amino acid substitutions that a given peptide can have relative to a sequence consisting of 5 to 15 consecutive amino acids in SEQ ID NO:42 can vary depending on the length of the peptide, and can range from 1 amino acid substitution for a peptide that is 6 amino acids in length to 10 amino acid substitutions for a peptide that is 15 amino acids in length, provided that the peptide comprises at least 5 consecutive amino acids from the amino acid sequence LSSTQAQQSX₁ (SEQ ID NO:1) that have not been substituted. Permissible amino acid substitutions include, but are not limited to, substitution with conservative amino acids and/or amino acid analogs.

Preferably, a peptide having one or more amino acid substitutions as compared to the corresponding sequence in SEQ ID NO:42 has 1, 2, 3, 4, 5 or more conservative amino acid substitutions as compared to the corresponding amino acid in SEQ ID NO:42.

In some embodiments, a peptide has an amino acid having an aromatic side chain (e.g., phenylalanine, tryptophan, or tyrosine) at the N-terminus and/or C-terminus of the peptide. In some embodiments, a peptide of the disclosure has an amino acid having an aromatic side chain at the C-terminus but not the N-terminus. In some embodiments, the peptide has a C-terminal tryptophan.

A peptide having amino acid substitutions as compared to the corresponding sequence in SEQ ID NO:42 can include one or more amino acid analogs (e.g., 1, 2, 3, 4, or 5 amino acid analogs). Generally, as used herein, an amino acid refers to a naturally occurring L-stereoisomer, and in some embodiments, each of the amino acids in the peptide is a naturally occurring L stereoisomer. An amino acid analog refers to a D-stereoisomer or an unnatural amino acid. For example, unnatural amino acids include, but are not limited to azetidinecarboxylic acid, 2-aminoadipic acid, 3-aminoadipic acid, β-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline.

The peptides can be entirely L-amino acids, entirely D-amino acids, or a mixture of L-amino acids and D-amino acids, with peptides that are entirely L-amino acids being preferred. Peptides containing two or more asymmetric carbon atoms can be any form of mixtures of enantiomers or diastereomers in any ratio.

The peptides can be in the form of salts, preferably comprising counterions that are pharmaceutically acceptable (e.g., chloride, sulfate, citrate, phosphate, acetate, sodium, potassium, calcium, magnesium). Peptide salts can be acid addition salts or a base addition salts. Exemplary acids that can be used to make an acid addition salt include hydrochloric acid, sulfuric acid, nitric acid, phosphoric acid, hydrobromic acid, perchloric acid, citric acid, succinic acid, maleic acid, fumaric acid, malic acid, tartaric acid, p-toluenesulfonic acid, benzenesulfonic acid, methanesulfonic acid, and trifluoroacetic acid. In some embodiments, the peptide is in the form of a hydrochloride salt. Exemplary bases that can be used to make a base addition salt include sodium hydroxide, potassium hydroxide, bases of alkali metals, such as lithium hydroxide, calcium hydroxide, and bases of alkaline earth metal salts such as magnesium hydroxide. Additional acids and bases that can be used to make pharmaceutically acceptable salts are described in Stahl and Wermuth, eds., 2008, Handbook of Pharmaceutical Salts: Properties, Selection and Use, Verlag Helvetica Chimica Acta, Zurich, Switzerland, the contents of which are incorporated herein by reference in their entireties.

The peptides be obtained, for example, by liquid phase peptide synthesis or solid phase peptide synthesis according to methods known in the art (e.g., as described in Benoiton, N., 2006, Chemistry of Peptide Synthesis, CRC Press, Boca Raton, FL; Howl, J., ed., 2005, Peptide Synthesis and Applications, Humana Press, Totowa, NJ; Chan and White, eds., 2000, Fmoc Solid Phase Synthesis: A Practical Approach, Oxford University Press, Oxford, UK). Custom peptide synthesis is also available commercially from numerous vendors (e.g., ABI Scientific (Sterling, VA); AnaSpec (Freemont CA); Pepscan, (Lelystad, Netherlands), Neo Scientific (Cambridge, MA); Sigma-Aldrich (St. Louis, MO)).

### 6.2.2. Peptide Conjugates

Peptide conjugates and salts thereof that can be used in the compositions and methods of the disclosure comprise a peptide moiety and a conjugate moiety. Attachment of a conjugate moiety to a peptide can provide, for example, improved water solubility, improved stability, and reduced clearance as compared to the non-conjugated peptide (Hamley, 2014, Biomacromolecules 15:1543-1559). Thus, peptide conjugates can in some instances be more suitable as therapeutic agents compared to their unconjugated counterparts. Exemplary peptide moieties are described in Section 6.2.2.1 and exemplary conjugate moieties are described in Section 6.2.2.2. It should be understood that when an embodiment described herein refers to a “peptide conjugate,” the embodiment encompasses the peptide conjugate per se as well as salts of the peptide conjugate even though the embodiment may not explicitly recite the expression “or salt thereof” or similar, unless required otherwise by context. It should be further understood that the term “peptide conjugate” as used herein does not encompass a peptide whose amino acid sequence corresponds to a contiguous amino acid sequence found in a naturally occurring peptide or protein, where a portion of the amino acid sequence is arbitrarily designated as a “peptide moiety” and another portion of the peptide sequence is arbitrarily designated as a “conjugate moiety.”

### 6.2.2.1. Peptide Moieties

Peptide moieties have an amino acid sequence that comprises or consists of at least 5 consecutive amino acids from the amino acid sequence LSSTQAQQSX₁ (SEQ ID NO:1), The peptide moiety is attached (i.e., covalently) to one or more conjugate moieties (e.g., 1, 2, 3, 4, or 5 conjugate moieties). The peptides described in Section 6.2.1 and WO 2018/047852, the contents of which are incorporated by reference in their entirety, can be used as a peptide moiety.

### 6.2.2.2. Conjugate Moieties

The peptide conjugates comprise one or more conjugate moieties (e.g., 1, 2, 3, 4, or 5 conjugate moieties) attached to the peptide moiety. The conjugate moiety or moieties can be attached to an N-terminal amino acid, a C-terminal amino acid, an amino acid that is neither an N-terminal amino acid or a C-terminal amino acid, or a combination thereof. For example, a peptide conjugate can comprise one conjugate moiety, preferably which is either attached to the N-terminal amino acid of the peptide moiety or attached to the C-terminal amino acid of the peptide moiety. As another example, a peptide conjugate can comprise two conjugate moieties, one of which is preferably attached to the N-terminal amino acid of the peptide moiety and the other of which is preferably attached to the C-terminal amino acid of the peptide moiety.

In embodiments in which the peptide conjugate comprises multiple conjugate moieties, each of the conjugate moieties can be the same, some of the conjugate moieties can be the same and others can be different, or all of the conjugate moieties can be different. For example, a peptide conjugate having two conjugate moieties can have two of the same conjugate moiety. Alternatively, a peptide conjugate having two conjugate moieties can have two different conjugate moieties. As another example, a peptide conjugate having three conjugate moieties can have three of the same conjugate moiety, three different conjugate moieties, or two of the same conjugate moiety and one different conjugate moiety.

A conjugate moiety can be attached to a peptide moiety, for example, at one of the peptide moiety's amino acid side chains, its backbone, its N-terminal amino group, or its C-terminal carboxylic acid group. For example, a conjugate moiety can be attached to an amino acid side chain to form a chemically modified amino acid, such as methionine sulfoxide, methionine sulfone, S-(carboxymethyl)-cysteine, S-(carboxymethyl)-cysteine sulfoxide and S-(carboxymethyl)-cysteine sulfone. Other side chain modifications include acylation of lysine ε-amino groups, N-alkylation of arginine, histidine, or lysine, and alkylation of glutamic or aspartic carboxylic acid groups. Conjugate moieties can be attached to the peptide backbone, for example to a nitrogen atom in the backbone (e.g., a methyl conjugate moiety can be introduced into a peptide conjugate's backbone by using an N-methyl amino acid to synthesize the peptide). Conjugate moieties can be attached to the N-terminal amino group of the peptide moiety to provide, for example, an N-terminus having an N-lower alkyl, N-di-lower alkyl, or N-acyl modifications. Conjugate moieties can be attached to the C-terminal carboxy group to provide, for example, a peptide conjugate having an amide, a lower alkyl amide, a dialkyl amide, or a lower alkyl ester at the C-terminus of the conjugate. A lower alkyl refers to a C₁-C₄ alkyl.

Exemplary conjugate moieties that can be used in the peptide conjugates include polymers, amine groups (e.g., amino (—NH₂), alkyl amino and dialkyl amino), acyls groups (e.g., formyl or acetyl), alkyl groups (e.g., C₁-C₄ alkyl), phosphate groups, lipids and sugars.

In some embodiments, at least one, more than one, or all of the conjugate moieties in the peptide conjugate comprise(s) a polymer. Exemplary polymers that can be used as conjugate moieties include polyethylene glycol, polyvinyl pyrrolidone, polylactic-co-glycolic acid, N-(2-hydroxypropyl) methacrylamide copolymer, polyglutamic acid, and polysaccharides. In some embodiments, at least one, more than one, or all of the conjugate moieties in the peptide conjugate comprise(s) a polyethylene glycol. In some embodiments, at least one, more than one, or all of the conjugate moieties in the peptide conjugate comprise(s) polyvinyl pyrrolidone. In some embodiments, at least one, more than one, or all of the conjugate moieties in the peptide conjugate comprise(s) polylactic-co-glycolic acid. In some embodiments, at least one, more than one, or all of the conjugate moieties in the peptide conjugate comprise(s) N-(2-hydroxypropyl) methacrylamide copolymer. In some embodiments, at least one, more than one, or all of the conjugate moieties in the peptide conjugate comprise(s) polyglutamic acid. In some embodiments, at least one, more than one, or all of the conjugate moieties in the peptide conjugate comprise(s) comprises a polysaccharide.

In some embodiments, at least one, more than one, or all of the conjugate moieties in the peptide conjugate comprise(s) an amine group. Exemplary amine groups include amino (—NH₂), alkyl amino, and dialkyl amino groups. The alkyl groups can be, for example, a C₁-C₄ alkyl. In some embodiments, at least one, more than one, or all of the conjugate moieties in the peptide conjugate comprise(s) an amino group. In some embodiments, at least one, more than one, or all of the conjugate moieties in the peptide conjugate comprise(s) an alkyl amino group. In some embodiments, at least one, more than one, or all of the conjugate moieties in the peptide conjugate comprise(s) a dialkyl amino group.

In some embodiments, at least one, more than one, or all of the conjugate moieties in the peptide conjugate comprise(s) an acyl group. Exemplary acyl groups include formyl groups and acetyl groups. In some embodiments, at least one, more than one, or all of the conjugate moieties in the peptide conjugate comprise(s) a formyl group. In some embodiments, at least one, more than one, or all of the conjugate moieties in the peptide conjugate comprise(s) an acetyl group.

In some embodiments, at least one, more than one, or all of the conjugate moieties in the peptide conjugate comprise(s) an alkyl group. In exemplary embodiments, the alkyl group is a lower alkyl group, such as methyl or ethyl. In some embodiments, at least one, more than one, or all of the conjugate moieties in the peptide conjugate comprise(s) a methyl group. In some embodiments, at least one, more than one, or all of the conjugate moieties in the peptide conjugate comprise(s) an ethyl group.

In some embodiments, at least one, more than one, or all of the conjugate moieties in the peptide conjugate comprise(s) a phosphate group, for example attached to the side chain of a serine, threonine, or tyrosine.

In some embodiments, at least one, more than one, or all of the conjugate moieties in the peptide conjugate comprise(s) a lipid.

In some embodiments, at least one, more than one, or all of the conjugate moieties in the peptide conjugate comprise(s) a sugar.

In some embodiments, the peptide conjugate comprises one or more of the peptide modifications described in PCT international publication nos. WO 2016/140277A1 or WO 2018/047852, the contents of which are incorporated herein by reference in their entireties.

Processes for attaching conjugate moieties to peptide moieties are known in the art and can be used to obtain the peptide conjugates described herein (e.g., as described in Basle et al., 2010, Chemistry & Biology 17:213-227; Benoiton, N., 2006, Chemistry of Peptide Synthesis, CRC Press, Boca Raton, FL; Ernst and Leumann, eds., 1995, Modern Synthetic Methods, Verlag Helvetica Chimica Acta, Basel, Switzerland: Hamley, 2014, Biomacromolecules 15:1543-1559; Lundblad, R., 1995, Techniques in Protein Modification, CRC Press, Boca Raton, FL). Custom synthesis of peptide conjugates is also commercially available from numerous vendors (e.g., ABI Scientific (Sterling, VA): AnaSpec (Freemont CA); Pepscan, (Lelystad, Netherlands), Neo Scientific (Cambridge, MA); Sigma-Aldrich (St. Louis, MO), variously offering, for example, peptides having N-terminal conjugate moieties such as an acetyl group, a formyl group, a fatty acid, and alkyl amino groups, peptides having C-terminal conjugate moieties such as an amido group, alkyl amino groups, and alkyl groups, peptides conjugated to fatty acids, peptides conjugated to polyethylene glycol, and peptides having a phosphate conjugate moiety (e.g., comprising phosphoserine, phosphothreonine, or phosphotyrosine)).

The peptide conjugates can be in the form of salts, preferably comprising counterions that are pharmaceutically acceptable (e.g., chloride, sulfate, citrate, phosphate, acetate, sodium, potassium, calcium, magnesium). The peptide conjugate salts can be an acid addition salt or a base addition salt. Exemplary acids that can be used to make an acid addition salt include hydrochloric acid, sulfuric acid, nitric acid, phosphoric acid, hydrobromic acid, perchloric acid, citric acid, succinic acid, maleic acid, fumaric acid, malic acid, tartaric acid, p-toluenesulfonic acid, benzenesulfonic acid, methanesulfonic acid, and trifluoroacetic acid. Exemplary bases that can be used to make a base addition salt include sodium hydroxide, potassium hydroxide, bases of alkali metals, such as lithium hydroxide, calcium hydroxide, and bases of alkaline earth metal salts such as magnesium hydroxide. Additional acids and bases that can be used to make pharmaceutically acceptable salts are described in Stahl and Wermuth, eds., 2008, Handbook of Pharmaceutical Salts: Properties, Selection and Use, Verlag Helvetica Chimica Acta, Zurich, Switzerland, the contents of which are incorporated herein by reference in their entireties.

### 6.3. Pharmaceutical Compositions

In some aspects, the disclosure provides pharmaceutical compositions comprising a peptide, a pharmaceutically acceptable salt thereof, a peptide conjugate, or a pharmaceutically acceptable salt thereof as described in Section 6.2. The pharmaceutical compositions are preferably formulated for oral administration. Pharmaceutical compositions can be prepared according to standard methods in the art (e.g., as described in Allen et al., eds., 2012, Remington: The Science and Practice of Pharmacy, 22^{nd} Edition, Pharmaceutical Press, London, UK).

In some aspects, the disclosure provides uses of a peptide, a pharmaceutically acceptable salt thereof, a peptide conjugate, or a pharmaceutically acceptable salt thereof as described in Section 6.2 in the manufacture of a medicament, for example, a medicament for treating a subject having or at risk of a disease or condition described in Section 6.4.

### 6.4. Methods of Treatment

The compounds and compositions of the disclosure can be used to treat or prevent various diseases, disorders and conditions. This disclosure provides therapeutic uses for the compounds and compositions disclosed herein, including for the treatment of diseases, disorders, and conditions amenable to treatment by vagus nerve stimulation and/or modulation of histamine and/or norepinephrine levels and/or modulation of the inflammatory cytokine TNF-α.

In some aspects, the disclosure provides a method for treating a subject suffering from or at risk of an inflammatory condition comprising administering to the subject a compound or composition of the disclosure in an amount effective to treat the subject. As would be understood by persons skilled in the art, various conditions have inflammatory aspects and can be considered inflammatory conditions, for example, inflammatory bowel disease, rheumatoid arthritis, and schizophrenia.

In some aspects, the disclosure provides a method for treating a subject suffering from or at risk of schizophrenia or psychosis comprising administering to the subject a compound or composition of the disclosure in an amount effective to treat the subject. In some embodiments, the subject is suffering from schizophrenia. In some embodiments, the subject exhibits psychotic behavior.

Schizophrenia is characterized by positive symptoms (e.g., delusions and hallucinations), negative symptoms, abnormalities in mood, and cognition deficits, and often leads to severe functional impairment (Yasui-Furukori, 2012, Drug Des Devel Therapy 6:107-115). The treating can comprise, for example, improving or slowing progression of one or more negative symptoms of schizophrenia or psychosis. Negative symptoms include asociality, anhedonia, alogia, affective flattening, apathy, avolition, blunted affect, anergia, apathy, depression, low mood, and cognitive impairment. The cognitive impairment can be, for example, a deficit in verbal working memory, spatial working memory, verbal fluency, verbal learning, or a combination thereof. Negative symptoms, and changes in such symptoms, can be assessed by various scales and scores, for example, Positive and Negative Syndrome Scale (PANSS) (Kay et al., 1987, Schizophr Bull. 13 (2): 261-76), Scale for the Assessment of Negative Symptoms (SANS) (Andreasen, 1982, Arch Gen Psychiatry 39 (7): 784-8), Personal and Social Performance (PSP) scale (Morosini et al., 2000, Acta Psychiatr Scand. 101 (4): 323-9), Clinical Global Impression Improvement (CGI-I) scale (Busner & Targum, 2007, Psychiatry (Edgmont). 4 (7): 28-37), and Clinical Global Impression Severity (CGI-S) scale (Busner & Targum, 2007, Psychiatry (Edgmont). 4 (7): 28-37). Treating a subject with a compound or composition of the disclosure can comprise improving or slowing worsening of one or more negative symptoms as measured by one or more of these scales or scores, or a portion thereof.

In another aspect, the disclosure provides a method for treating a subject suffering from a neurodegenerative disease such as Parkinson's disease, Alzheimer's disease, or amyotrophic lateral sclerosis (ALS), comprising administering to the subject a compound or composition of the disclosure in an amount effective to treat the subject. The treating can comprise improving or slowing progression of one or more negative symptoms of the neurodegenerative disease, for example asociality, anhedonia, alogia, affective flattening, apathy, avolition, blunted affect, anergia, depression, low mood, and cognitive impairment. Negative symptoms, and changes in such symptoms, can be assessed by various scales and scores, for example those described in the preceding paragraph.

In some aspects, the disclosure provides a method for treating a subject suffering from Parkinson's disease comprising administering to the subject a compound or composition of the disclosure in an amount effective to treat the subject. The treating can comprise improving or slowing progression of one or more non-motor symptoms of Parkinson's disease. Non-motor symptoms of Parkinson's disease include sensory symptoms (e.g., numbness, restlessness, pain, chest discomfort, anosmia), cognitive symptoms (e.g., mood changes, depression, anxiety, panic attacks, tiredness, confusion, slowed thinking), and autonomic symptoms (e.g., hot/cold sensations, bladder problems, sweating, abdominal discomfort, constipation, sialorrhea (drooling or excessive salivation), frequent urination and/or urgency, erectile dysfunction) (see, e.g., Barone et al., 2009, Mov Disord. 24 (11): 1641-9). Non-motor symptoms, and changes in such symptoms, can be assessed by various scales and scores, for example, the Non-motor Symptoms Scale (NMSS) (Chaudhuri et al., 2007, Movement Disorders 22 (13): 1901-11). Movement Disorder Society Unified Parkinson's Disease Rating Scale (MDS-UPDRS) non-motor symptoms score (Goetz et al., International Parkinson and Movement Disorder Society, Aug. 13, 2019 update: www.movementdisorders.org/MDS-Files1/PDFs/MDS-UPDRS_English_FINAL.pdf)). Fatigue Severity Scale (FSS) score (Krupp et al., 1989, Arch Neurol. 46 (10): 1121-3), Beck's Depression Inventory II (BDI-II) score (Beck, Steer, & Brown, 1996), Beck Anxiety Inventory (BAI) score (Beck et al., 1988, J Consult Clin Phychol 56 (6): 893-7), Montreal Cognitive Assessment (MoCA) score (Nasreddine et al., 2005. J Am Geriatr Soc. 53 (4): 695-9), scores on Cogstate test(s) (cogstate.com), Clinical Global Impression Improvement (CGI-I) overall score or non-motor symptoms score (Busner & Targum, 2007, Psychiatry (Edgmont). 4 (7): 28-37), Clinical Global Impression Severity (CGI-S) overall or non-motor symptoms score (Busner & Targum, 2007, Psychiatry (Edgmont). 4 (7): 28-37), and Parkinson's Disease Questionnaire-39 (PDQ-39) (Jenkinson et al., 1997, Age Ageing. 26 (5): 353-7). Cogstate tests include the following computerized cognitive assessments: Behavioral Pattern Separation Object test; Continuous Paired Associate Learning Test; Face Name Associative Memory Exam; Groton Maze Learning Test; Identification Test; International Daily Symbol Substitution Tests (medicines or symbols); International Shopping List Test; One Back Test; One Card Learning Test; Sustained Attention Test; Sustained Attention to Response Test; and Two Back Test.

In another aspect, the disclosure provides a method for treating a subject suffering from gastrointestinal disease or condition comprising administering to the subject a compound or composition of the disclosure in an amount effective to treat the subject.

In another aspect, the disclosure provides a method for treating a subject suffering from irritable bowel syndrome comprising administering to the subject a compound or composition of the disclosure in an amount effective to treat the subject.

In another aspect, the disclosure provides a method for treating a subject suffering from inflammatory bowel disease comprising administering to the subject a compound or composition of the disclosure in an amount effective to treat the subject.

In another aspect, the disclosure provides a method for treating a subject suffering from ulcerative colitis comprising administering to the subject a compound or composition of the disclosure in an amount effective to treat the subject.

In another aspect, the disclosure provides a method for treating a subject suffering from Crohn's disease comprising administering to the subject a compound or composition of the disclosure in an amount effective to treat the subject.

In another aspect, the disclosure provides a method for treating a subject suffering from constipation comprising administering to the subject a compound or composition of the disclosure in an amount effective to treat the subject.

In another aspect, the disclosure provides a method for treating a subject suffering from pain comprising administering to the subject a compound or composition of the disclosure in an amount effective to treat the subject.

In another aspect, the disclosure provides a method for treating a subject suffering from visceral pain comprising administering to the subject a compound or composition of the disclosure in an amount effective to treat the subject.

In another aspect, the disclosure provides a method for treating a subject suffering from rheumatoid arthritis comprising administering to the subject a compound or composition of the disclosure in an amount effective to treat the subject.

In another aspect, the disclosure provides a method for treating a subject suffering from migraine comprising administering to the subject a compound or composition of the disclosure in an amount effective to treat the subject.

In another aspect, the disclosure provides a method for treating a subject suffering from headache comprising administering to the subject a compound or composition of the disclosure in an amount effective to treat the subject.

In another aspect, the disclosure provides a method for treating a subject suffering from substance abuse (e.g., substance use disorder such as drug use disorder, polysubstance use disorder, alcohol use disorder, nicotine use disorder, or tobacco use disorder) comprising administering to the subject a compound or composition of the disclosure in an amount effective to treat the subject. In some embodiments, the subject has drug use disorder. In some embodiments, the subject has polysubstance use disorder. In some embodiments, the subject has alcohol use disorder. In some embodiments, the subject has nicotine use disorder. In some embodiments, the subject has tobacco use disorder.

In another aspect, the disclosure provides a method for treating a subject suffering from drug addiction comprising administering to the subject a compound or composition of the disclosure in an amount effective to treat the subject. Examples of drug addiction include opioid addiction, for example addiction to morphine, heroin, oxycodone, or fentanyl; cocaine addiction; and benzodiazepine addiction such as addiction to diazepam, alprazolam, or clonazepam.

In another aspect, the disclosure provides a method for treating a subject suffering from a seizure disorder comprising administering to the subject a compound or composition of the disclosure in an amount effective to treat the subject. In some embodiments, the seizure disorder is epilepsy. In some embodiments, the seizure disorder is an orphan epilepsy (see, e.g., Perucca et al., 2020. Lancet 19:544-556).

In another aspect, the disclosure provides a method for treating a subject suffering from major depressive disorder comprising administering to the subject a compound or composition of the disclosure in an amount effective to treat the subject.

In another aspect, the disclosure provides a method for treating a subject suffering from atypical depression comprising administering to the subject a compound or composition of the disclosure in an amount effective to treat the subject.

In another aspect, the disclosure provides a method for treating a subject suffering from a major depressive episode (MDE) (e.g., atypical MDE) comprising administering to the subject a compound or composition of the disclosure in an amount effective to treat the subject.

In another aspect, the disclosure provides a method for treating a subject suffering from depression in the presence of a neurodegenerative disease (e.g., Parkinson's disease, Alzheimer's disease, or ALS) comprising administering to the subject a compound or composition of the disclosure in an amount effective to treat the subject. Depression in subjects having a neurodegenerative disease is typically resistant to traditional anti-depressant therapy (Hussain et al., 2020, Cureus 12 (11): e11613).

In another aspect, the disclosure provides a method for treating a subject suffering from treatment-resistant depression comprising administering to the subject a compound or composition of the disclosure in an amount effective to treat the subject.

In another aspect, the disclosure provides a method for treating a subject suffering from cognitive impairment comprising administering to the subject a compound or composition of the disclosure in an amount effective to treat the subject.

In another aspect, the disclosure provides a method for treating a subject suffering from Alzheimer's disease comprising administering to the subject a compound or composition of the disclosure in an amount effective to treat the subject. In another aspect, the disclosure provides a method for treating a subject suffering from COVID-19 related cognitive impairment and/or depression comprising administering to the subject a compound or composition of the disclosure in an amount effective to treat the subject.

In another aspect, the disclosure provides a method for treating a subject suffering from ADHD comprising administering to the subject a compound or composition of the disclosure in an amount effective to treat the subject.

In another aspect, the disclosure provides a method for treating a subject suffering from an autism spectrum disorder comprising administering to the subject of the disclosure a compound or composition in an amount effective to treat the subject.

In another aspect, the disclosure provides a method for treating a subject suffering from a pervasive developmental disorder, such as Asperger syndrome or Rett syndrome, comprising administering to the subject a compound or composition of the disclosure in an amount effective to treat the subject.

In another aspect, the disclosure provides a method for treating a subject suffering from pervasive developmental disorders not otherwise specified comprising administering to the subject a compound or composition of the disclosure in an amount effective to treat the subject.

In another aspect, the disclosure provides a method for treating a subject suffering from multiple sclerosis comprising administering to the subject a compound or composition of the disclosure in an amount effective to treat the subject.

In another aspect, the disclosure provides a method for treating a subject suffering from PTSD comprising administering to the subject a compound or composition of the disclosure in an amount effective to treat the subject.

In another aspect, the disclosure provides a method for treating a subject suffering from a sleep disorder comprising administering to the subject a compound or composition of the disclosure in an amount effective to treat the subject.

In another aspect, the disclosure provides a method for treating a subject suffering from insomnia comprising administering to the subject a compound or composition of the disclosure in an amount effective to treat the subject.

In another aspect, the disclosure provides a method for treating a subject suffering from daytime fatigue comprising administering to the subject a compound or composition of the disclosure in an amount effective to treat the subject.

In another aspect, the disclosure provides a method for treating a subject suffering from REM sleep behavior disorder comprising administering to the subject a compound or composition of the disclosure in an amount effective to treat the subject.

In another aspect, the disclosure provides a method for improving sleep in a subject comprising administering to the subject a compound or composition of the disclosure in an amount effective to improve the subject's sleep.

In another aspect, the disclosure provides a method for improving cognitive function of a subject comprising administering to the subject a compound or composition of the disclosure in an amount effective to improve cognitive function. The subject may be a healthy subject, e.g., one that does not suffer from a condition described herein.

In another aspect, the disclosure provides a method for improving mood of a subject comprising administering to the subject a compound or composition of the disclosure in an amount effective to improve mood. The subject may be a healthy subject, e.g., one that does not suffer from a condition described herein.

In another aspect, the disclosure provides a method for improving or slowing worsening of an overall Patient Health Questionnaire-9 (PHQ-9) score of a subject comprising administering to the subject a compound or composition of the disclosure in an amount effective to improve or slow worsening of the subject's overall PHQ-9 score. The subject may be a healthy subject, e.g., one that does not suffer from a condition described herein.

In another aspect, the disclosure provides a method for improving or slowing worsening of a subject's score on one or more of questions of PHQ-9, such as PHQ-9 questions 1, 2, 3, or 6, comprising administering to the subject a compound or composition of the disclosure in an amount effective to improve or slow worsening of the subject's score on the PHQ-9 question(s).

In another aspect, the disclosure provides a method for improving or slowing worsening of one or more of the CogState Groton Maze Learning Test (GMLT), the CogState Identification Test (IDN), or the CogState One Card Learning Test (OCL) results of a subject, comprising administering to the subject a compound or composition of the disclosure in an amount effective to improve or slow worsening of the subject's result(s) on the GMLT, IDN, and/or OCL. The subject may be a healthy subject, e.g., one that does not suffer from a condition described herein.

In another aspect, the disclosure provides a method for improving or slowing worsening of at least one of the alpha band power or the gamma band power of an electroencephalogram (EEG) of a subject. The subject may be a healthy subject, e.g., one that does not suffer from a condition described herein.

In some embodiments of the methods described herein, the subject has an elevated TNF-α level, e.g., relative to a reference range obtained from healthy subjects. For example, in some embodiments, a subject having an elevated TNF-α level has a serum TNF-α level greater than 5.6 pg/ml, for example as measured by a sandwich immunoassay (e.g., the Eurofins Viracor TNF-α serum test; www.eurofins-viracor.com/clinical/test-menu/1220-tnf-alpha-tnf-a-serum.

The subjects of the methods described herein are preferably mammals, e.g., humans or domestic pets (e.g., cat, dog). Subjects can be of any age, but are preferably adults (e.g., a human subject who is 18 years old or more, 25 years old or more, 35 years old or more, 45 years old or more, 55 years old or more, etc.). In some embodiments, the subject is elderly (e.g., a human subject who is 65 years old or more, 70 years old or more, 75 years old or more, or 80 years old or more).

The compounds and pharmaceutical compositions containing them can be administered orally, topically, rectally, or parenterally, with oral administration being preferred. The method of administration can vary depending on the condition and age of the subject.

Appropriate daily dosages of the compounds can range from 0.005 mg/kg to 500 mg/kg of body weight per day (e.g., 0.005 mg/kg to 100 mg/kg, 0.005 mg/kg to 30 mg/kg, 0.005 mg/kg to 1 mg/kg, 0.01 mg/kg to 30 mg/kg, 0.01 mg/kg to 3 mg/kg. 0.01 mg/kg to 1 mg/kg, 0.02 mg/kg to 5 mg/kg, 0.02 mg/kg to 2 mg/kg. 0.02 mg/kg to 1 mg/kg). Alternatively, the compounds can be administered at a fixed dose ranging from 0.1 mg to 50 g per day (e.g., 0.1 mg to 10 g, 0.1 mg to 3 g, 0.1 mg to 100 mg, 0.1 mg to 1 mg, 0.3 mg to 3 g. 0.3 mg to 100 mg, 20 mg to 2 g, 20 mg to 1 g, 0.5 g to 2 g, 0.5 g to 1 g, or 1 g to 2 g). For administration of a pharmaceutical composition, an amount of the pharmaceutical composition can be administered that contains an amount of the compound that is within one of the foregoing ranges. Exemplary daily doses of the compounds described herein (e.g., the peptide LSSTQAQQSY (SEQ ID NO:2) or a salt thereof, include 60 mg/day, 180 mg/day, and 540 mg/day. A daily dose can be administered as a single dose, e.g., a single dose of 60 mg, 180 mg, or 540 mg, or multiple doses.

### 7. EXAMPLES

### 7.1. Example 1: LSSTQAQQSY (SEQ ID NO:2) Brain Microdialysis Study

A study was performed to determine the CNS neurotransmitter responses elicited by oral dosing with the peptide LSSTQAQQSY (SEQ ID NO:2).

Male C57Bl/6 mice were fitted with microdialysis probes in the prefrontal cortex (which modulates sensory processing, memory, and emotions) or striatum (required for behavioral reinforcement by natural rewards). Animals were orally administered a single 3 mg/kg dose of peptide or vehicle. Brain dialysate samples were collected before administration and every 30 minutes for four hours after. Dopamine, norepinephrine, serotonin, histamine, glutamate, GABA, and glycine concentrations in dialysate samples were determined by HPLC-MS.

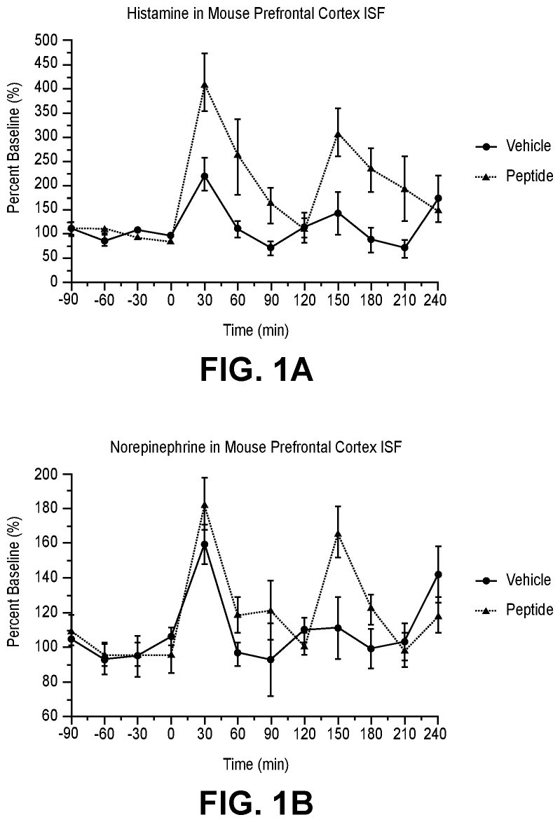

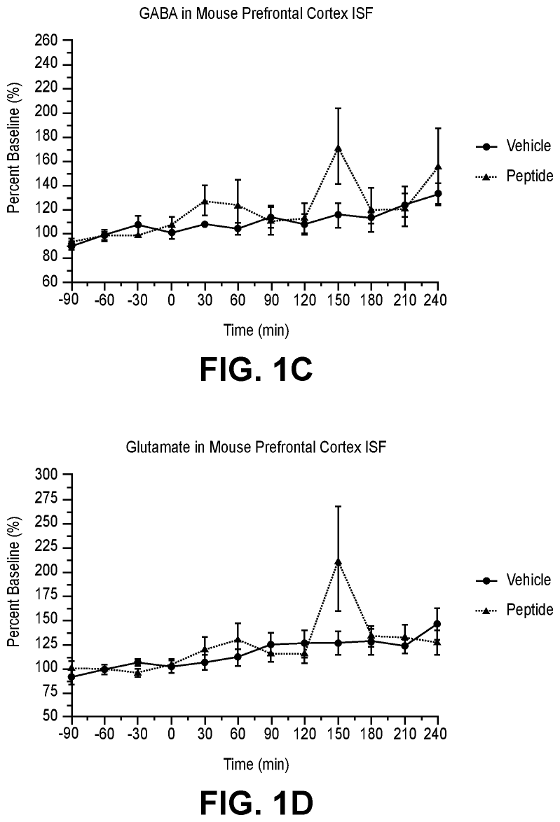

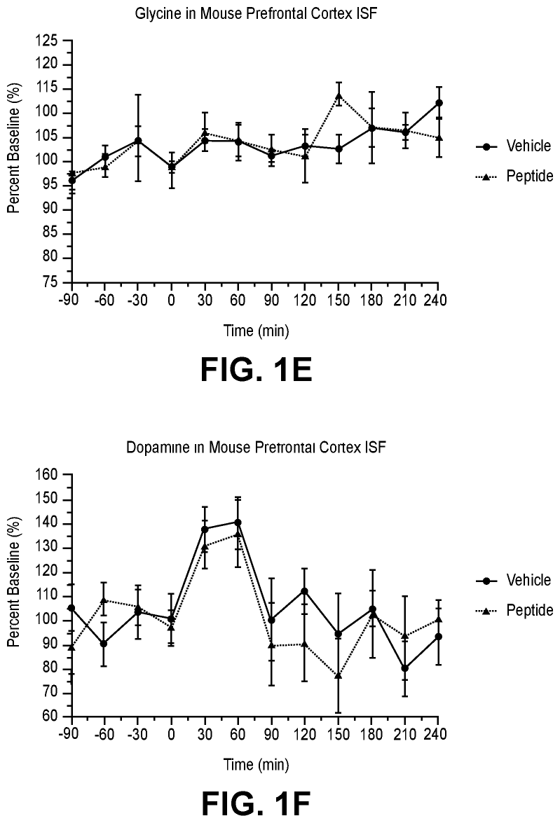

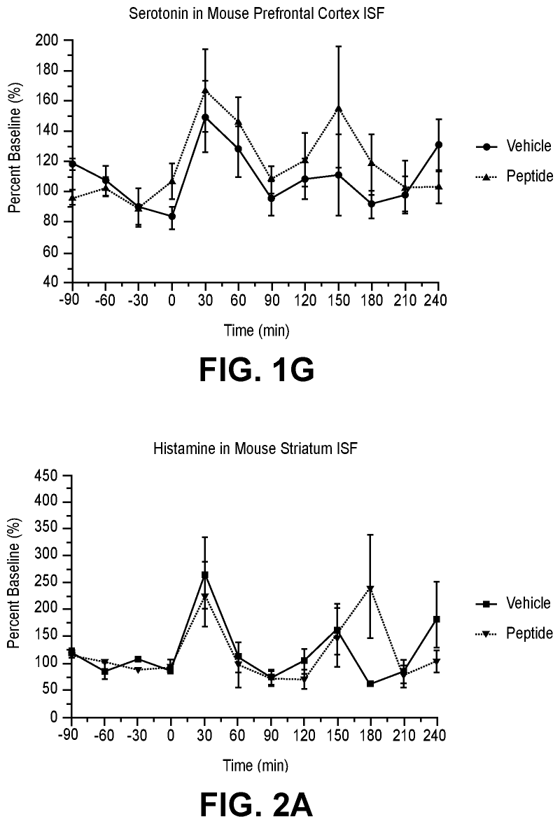

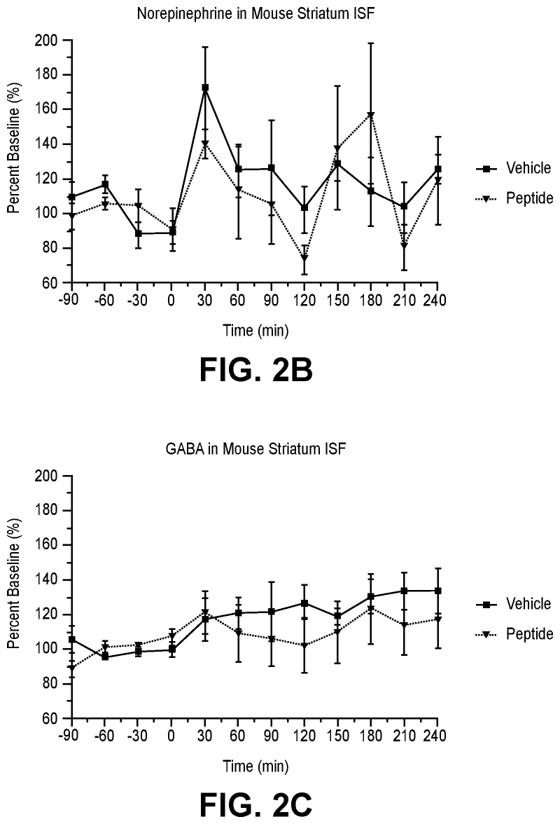

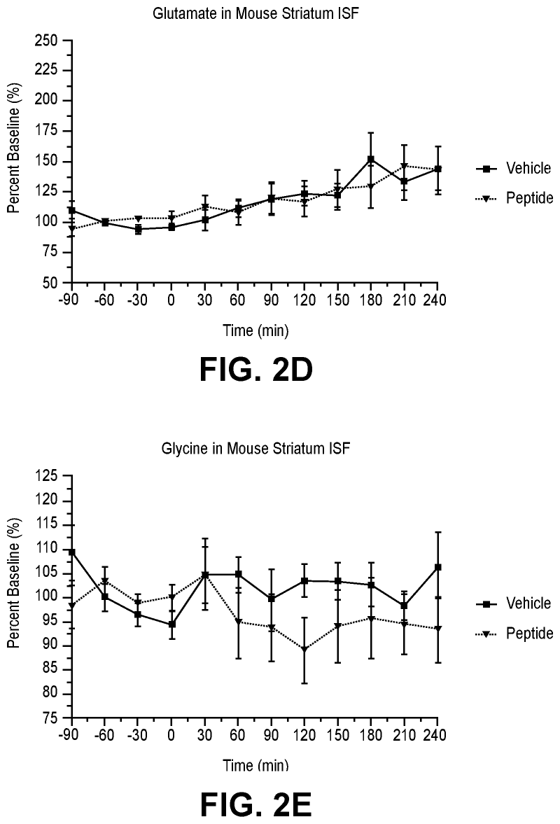

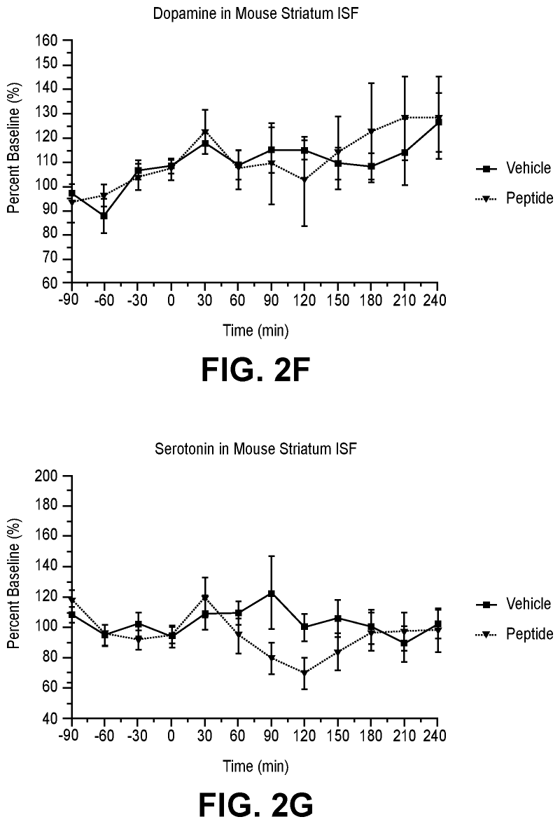

Results are shown in FIGS. 1A-1G and FIGS. 2A-2G.

Statistically significant changes in histamine and norepinephrine in the prefrontal cortex were observed. No significant changes were observed with other neurotransmitters in the prefrontal cortex and all evaluated neurotransmitters in striatum.

### 7.2. Example 2: Inflammation Suppression by LSSTQAQQSY (SEQ ID NO:2)

Vagus nerve stimulation has been reported to suppress lipopolysaccharide (LPS)-induced increases in serum levels of the inflammatory cytokine TNF-α (see, e.g., Tarnawski et al., 2018, Front. Immunol. 9:2648; Komegae et al., 2018, Brain Behav Immun. 73:441-449). As the peptide LSSTQAQQSY (SEQ ID NO:2) is believed to act via the vagus nerve, the ability of the peptide to suppress TNF-α following LPS injection was assessed.

Mice were administered LPS at a dose of 10 μg/kg, i.p. The peptide LSSTQAQQSY (SEQ ID NO:2) was administered twice to the mice at 0.3 mg/kg, 3 mg/kg, or 10 mg/kg, p.o., 2.5 and 3.5 hours the after LPS administration. Blood was collected 4 hours after the LPS administration. Plasma levels of TNF-α were subsequently measured by ELISA.

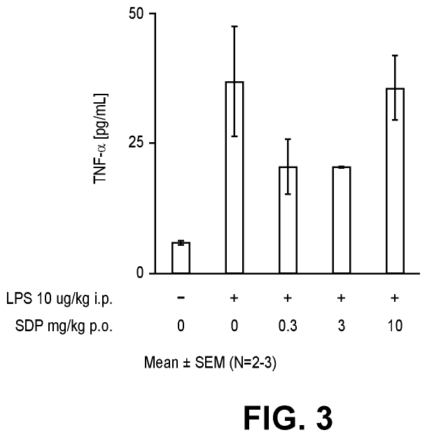

Results are shown in FIG. 3. A trend of TNF-α suppression was observed in animals administered the peptide following in vivo challenge with LPS, indicating an anti-inflammatory effect of the peptide.

### 7.3. Example 3: Phase 2 Study in Subjects with Schizophrenia (Prophetic)

A Phase 2, 4-week, randomized, placebo-controlled, crossover study with the peptide LSSTQAQQSY (SEQ ID NO:2) in subjects having schizophrenia with predominantly negative symptoms and cognitive deficits is performed.

The study has 4-week treatment periods, with a 7-day washout between treatment periods, comparing the peptide to placebo.

The study population includes men and women, age 18 to 65, with schizophrenia (DSM-5/SCID-5-CT) having predominantly negative symptoms and cognitive deficits.

The primary outcome measure is the mean change from baseline in the Positive and Negative Syndrome Scale (PANSS) negative symptom factor score.

Secondary outcome measures include mean change from baseline in: Assessment of Negative Symptoms (SANS) score; Personal and Social Performance (PSP) total score; PANSS total score; PANSS factor scores: PANSS subscale scores; Clinical Global Impression Improvement (CGI-I) overall and negative symptoms rating score and Clinical Global Impression Severity (CGI-S) overall and negative symptoms rating score. Secondary outcome measures also include percentage of participants with response, as assessed by: PANSS negative symptom factor score; CGI-I overall and negative symptoms rating score; CGI-S overall and negative symptoms rating score.

Exploratory outcome measures include brain activity and cognitive-behavioral task performance using a combination of functional magnetic resonance imaging (fMRI) utilizing memory activation tasks, arterial spin labeling (ASL-MRI), and population PK.

Treatment with the peptide improves or slows the progression of one or more negative symptoms and/or cognitive deficits in subjects having schizophrenia with predominantly negative symptoms and cognitive deficits.

### 7.4. Example 4: Phase 2 Study in Subjects with Parkinson's Disease and Predominantly Non-Motor Symptoms (Prophetic)

A Phase 2, 6-week, randomized, placebo-controlled, crossover study with the peptide LSSTQAQQSY (SEQ ID NO:2) in subjects having Parkinson's disease with predominantly non-motor symptoms is performed.

The study has 6-week treatment periods, with a 7-day washout between treatment periods, comparing the peptide to placebo.

The study population includes men and women, ages 40 and older, with Parkinson's disease (DSM-5/ICD-10) having predominantly non-motor symptoms.

The primary outcome measure is the mean change from baseline in the Non-Motor Symptoms Scale (NMSS) score.

Secondary outcome measures include mean change from baseline in: non-motor symptoms of the Movement Disorder Society Unified Parkinson's Disease Rating Scale (MDS-UPDRS) (e.g., part I, cognition, depression, anxiety fatigue): motor symptoms (e.g., MDS-UPDRS, part II, III, IV); Fatigue Severity Scale (FSS) scores; Beck's Depression Inventory II (BDI-II) scores; Beck Anxiety Inventory (BAI) scores; Montreal Cognitive Assessment (MoCA) scores: Cogstate Scores; CGI-I overall and non-motor symptoms rating scores: CGI-S overall and non-motor symptoms rating scores; Parkinson's Disease Questionnaire-39 (PDQ-39). Exploratory outcome measures include brain activity and cognitive-behavioral task performance using a combination of functional magnetic resonance imaging (fMRI) utilizing memory activation tasks, arterial spin labeling (ASL-MRI), and population PK.

Treatment with the peptide improves or slows the progression of one or more non-motor symptoms in subjects having Parkinson's disease with predominantly non-motor symptoms.

### 7.5. Example 5: Effect of LSSTQAQQSY (SEQ ID NO:2) on Scopolamine-Induced Memory Impairment in Mice

### 7.5.1. Materials

### 7.5.1.1. Animals

Male ICR mice, aged 4-5 weeks were used in the study.

### 7.5.1.2. Drugs

The peptide LSSTQAQQSY (SEQ ID NO:2) and Donepezil Hydrochloride were used as study drugs.

### 7.5.1.3. Study Instruments

The study was performed in step-down inhibitory avoidance equipment with dimensions 12 cm×2 cm×18 cm in each chamber (Model: YLS-3T, Shandong Yiyan Technology Co., LTD., China). The bottom of each chamber was covered with a copper grid that can be electrified. A rubber platform was placed in the right corner of each chamber.

### 7.5.2. Methods

### 7.5.2.1. Drug Administration

Male ICR mice in each group were treated by oral gavage once a day for 21 days with the vehicle, Donepezil Hydrochloride (1.5 mg/kg), and the peptide LSSTQAQQSY (SEQ ID NO:2) (0.3, 10 mg/kg), respectively. Two hours after the last administration, scopolamine hydrobromide (2 mg/kg) was injected intraperitoneally to induce memory impairment.

### 7.5.2.2. Animal Groups

Fifty male ICR mice were divided into 5 groups according to the average of body weight, 10 mice in each group, as detailed in Table 1.

TABLE 1

| | | Dose | Number of |
|---|---|---|---|
| No. | Group | (mg/kg) | mice/Group |
| 1 | Vehicle | 0.5% CMC-Na | 10 |
| 2 | Model (Scopolamine | 0.5% CMC-Na | 10 |
| | alone) | | |
| 3 | Donepezil | 1.5 | 10 |
| | Hydrochloride | | |
| 4 | LSSTQAQQSY | 10 | 10 |
| | (SEQ ID NO:2) | | |
| 5 | LSSTQAQQSY | 0.3 | 10 |
| | (SEQ ID NO:2) | | |

### 7.5.2.3. Step-Down Inhibitory Avoidance Evaluation

Training period: The mice were intraperitoneally injected with scopolamine hydrobromide solution at a dose of 2 mg/kg for the Model, Donepezil Hydrochloride and LSSTQAQQSY (SEQ ID NO:2) groups, while the mice in the vehicle group were intraperitoneally injected with the same volume of 0.9% NaCl solution. Thirty minutes after injection of scopolamine hydrobromide solution, the mice were put into a step-down chamber for 5 minutes for adaption, then the mice received a foot shock (82 v voltage with 2 mA current) during the training session. The mice could jump onto and down the rubber stands during the shock freely.

Evaluation period: 24 h after the training period, the mice were placed on the rubber platform and evaluated for 5 minutes. The latency of the first jump from the platform to the copper grid was observed and counted, and the latency in seconds was analyzed to evaluate memory improvement.

### 7.5.2.4. Statistical Analyses

All results were given as mean±SEM, and the data differences between groups were analyzed by t-test for the paired comparisons using the GraphPad Prism 8 statistical package (GraphPad Software Inc., San Diego, CA, USA). P<0.05 was considered statistically significant.

### 7.5.3. Results

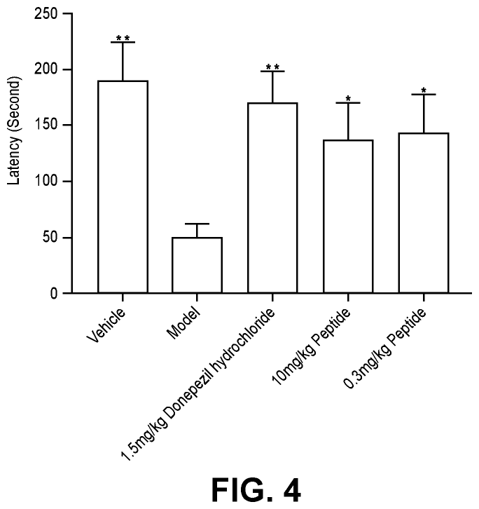

The latency in the model group was significantly shorter than the vehicle group (P<0.01). The latency in the positive control 1.5 mg/kg Donepezil Hydrochloride group was significantly longer than the model group (P<0.01). The 0.3 mg/kg and 10 mg/kg peptide treatment groups all extended their latencies, which was significantly different from the model group (P<0.05). The results are shown in FIG. 4. These results indicate that LSSTQAQQSY (SEQ ID NO:2) improved memory impairment induced by scopolamine in mice, indicating that LSSTQAQQSY (SEQ ID NO:2) and related peptides have potential for treating Alzheimer's disease and cognitive impairment associated with other diseases.

### 7.6. Example 6: LSSTQAQQSY (SEQ ID NO:2) Target Characterization

Several screens were performed to characterize the molecular target of LSSTQAQQSY (SEQ ID NO:2). Initial screens including screening of Trp channels (TRPA1, TRPV1), nutrient and metabolite sensing GPCRs (FFAR1, FFAR2, FFAR3, FFAR4, GPR119, CaS, GPR142, GPBAR1 and LPA5) and taste receptors; a full genome-wide binding-based screen of all cell surface-expressed receptors (˜6K receptors); and a large screen of 168 GPCRs for agonist or antagonist activity. The initial screens were negative or provided non-reproducible hits.

Subsequently, a more universal and more sensitive GPCR screening strategy was employed. In the screen, three possible targets were identified: GNRHR, AVPR1A, and AVPR2. Studies were then performed to identify and confirm the target.

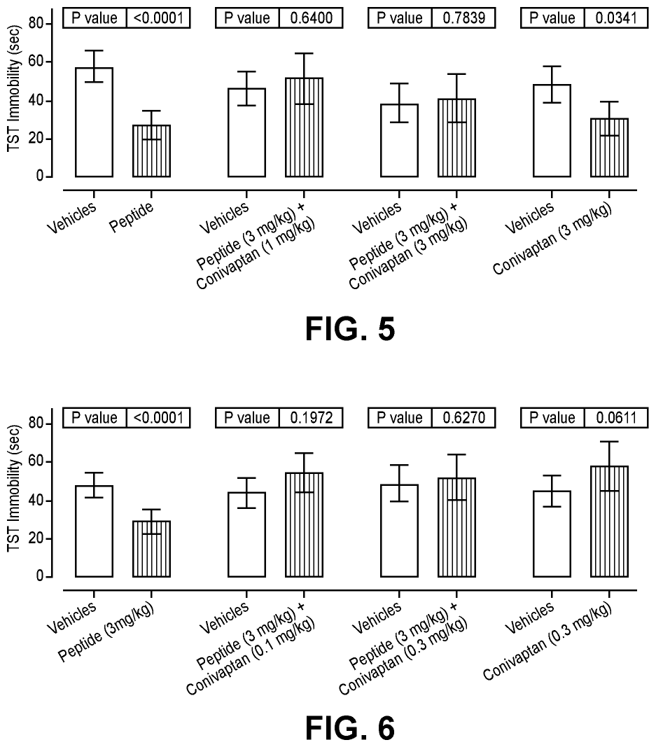

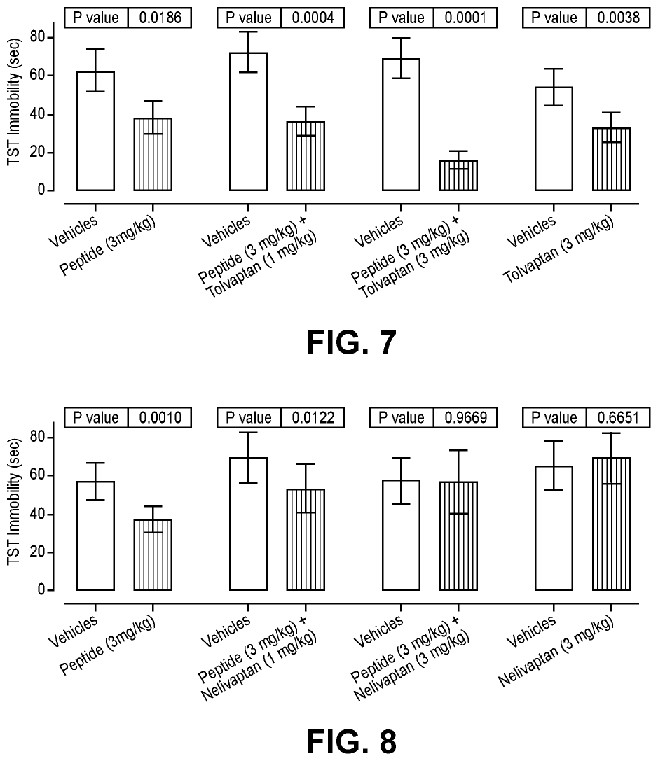

It was found that the effect of LSSTQAQQSY (SEQ ID NO:2) was blocked by the AVPR1A/AVPR2 inhibitor conivaptan in a tail suspension test (TST) (see, WO 2016/140277), suggesting that the primary target of LSSTQAQQSY (SEQ ID NO:2) is either AVPR1A or AVPR2 (FIGS. 5-6). It was found that the effect of LSSTQAQQSY (SEQ ID NO:2) was not blocked by the AVPR2 inhibitor tolvaptan (FIG. 7), suggesting that the primary target of the peptide is not AVPR2. It was also found that the effect of LSSTQAQQSY (SEQ ID NO:2) was blocked by the AVPR1A/B inhibitor nelivaptan (FIG. 8), indicating that the primary target of the peptide is AVPR1A.

The results of the studies are summarized in Table 2.

TABLE 2

| | | Peptide activity | Peptide | Peptide | |
|---|---|---|---|---|---|
| | | blocked by | activity | activity | |
| | | conivaptan | blocked by | blocked by | |
| | Activated by | (AVPR1A/ | tolvaptan | nelivaptan | |
| | peptide in | AVPR2 | (AVPR2 | (AVPR1A/1B | |
| Candidate | in vitro | inhibitor) | inhibitor) | inhibitor) | Likely |
| receptor | screens? | in vivo? | in vivo? | in vivo? | target |
| AVPR1A | Yes | Yes | NA | Yes | Yes |
| AVPR1B | No | NA | NA | Yes | No |
| AVPR2 | Yes | Yes | No | NA | No |

### 7.7. Example 7: Mood and Cognition Improvement in Healthy Subjects in Phase 1a/1b Study

A Phase 1a/1b, randomized, placebo-controlled, crossover study with the peptide LSSTQAQQSY (SEQ ID NO:2) in healthy subjects was performed. The study population included men and women in good general health.

A single ascending dose (SAD) group included four cohorts receiving 60 mg/day (cohorts SAD1 and SAD2). 180 mg/day (cohort SAD3), or 540 mg/day (cohort SAD4) of the peptide or placebo. A multiple ascending dose (MAD) group included three cohorts receiving 60 mg/day (cohort MAD1), 180 mg/day (cohort MAD2), or 540 mg/day (cohort MAD3) of the peptide or placebo for seven days. All doses were administered orally.

### 7.7.1. Results

### 7.7.1.1. Safety

A few members of each cohort experienced mild adverse effects, generally gastrointestinal upset or throat irritation. Vital signs, electrocardiography (ECG) data, and physical examinations were normal for all participants throughout the study.

### 7.7.1.2. Patient Questionnaires

Patient Health Questionnaire-9 (PHQ-9), a standard questionnaire for assessing depression severity, was administered to the subjects in cohorts MAD1-MAD3 about four hours after each of the placebo and peptide administration periods. Each of the nine questions asks the subject how often in the previous two weeks he or she has/had the following:

| Question # | |
|---|---|
| 1 | Little interest or pleasure in doing things? |
| 2 | Feeling down, depressed, or hopeless? |
| 3 | Trouble falling or staying asleep, or sleeping too much? |
| 4 | Feeling tired or having little energy? |
| 5 | Poor appetite or overeating? |
| 6 | Feeling bad about yourself-or that you are a failure or have let yourself or |
| | your family down? |
| 7 | Trouble concentrating on things, such as reading the newspaper or watching |
| | television? |
| 8 | Moving or speaking so slowly that other people could have noticed? Or the |
| | opposite-being so fidgety or restless that you have been moving around a lot |
| | more than usual? |
| 9 | Thoughts that you would be better off dead or of hurting yourself in some way? |

Each question is answered on a 0 to +3 scale, with 0 for not at all, +1 for several days, +2 for more than half the days, and +3 for nearly every day.

Whether the peptide led to improvement over placebo in each PHQ-9 question was assessed.

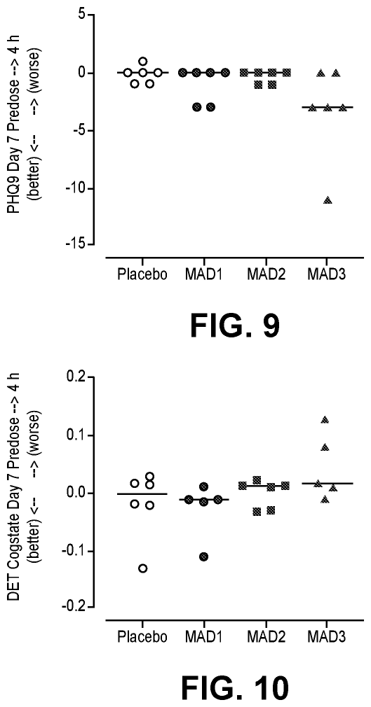

Preliminary data indicate that administration of the peptide led to improvement in measures of mood, depression, sleep, awareness, and/or alertness in cohort MAD3 (improvement in answers to PHQ-9 questions 1-3 and 6, with overall PHQ-9 score summarized in FIG. 9).

### 7.7.1.3. CogState

The subjects in cohorts MAD1-MAD3 also underwent cognitive assessments (CogState Ltd., Melbourne, Vic., Australia): 1) Detection Test (DET), an assessment of psychomotor function; 2) Groton Maze Learning Test (GMLT), an assessment of executive function; 3) Identification Test (IDN), an assessment of attention; and 4) One Card Learning Test (OCL), an assessment of visual learning. The cognitive assessments were performed at essentially the same times as administration of the PHQ-9 questionnaire to the subjects. Whether the peptide led to improvement over placebo in the four cognitive assessments was qualitatively determined.

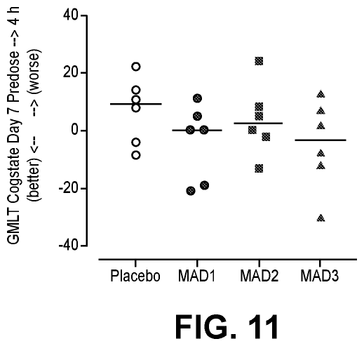

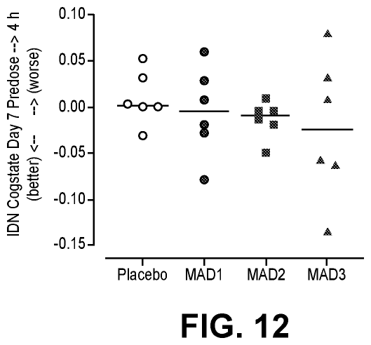

Preliminary data indicate that the peptide did not have a significant impact on psychomotor function in healthy subjects (FIG. 10). Preliminary data further indicate a positive trend in GMLT (FIG. 11), IDN (FIG. 12), and OCL (not shown) after seven days of treatment.

In conclusion, preliminary data from the study indicate that treatment with the peptide improved executive function, attention, and visual learning in the subjects.

### 7.7.1.4. Alpha and Gamma Electroencephalography (EEG) Bands

Various bands observed in EEG are associated with various states of mental activity, generally as summarized on the following table:

| Band | Frequency | |
|---|---|---|
| name | Range (Hz) | Mental Activity |
| Delta | 1.5-6 | Sleep, dreaming |
| Theta | 6-8.5 | Drowsiness |
| Alpha | 8.5-12.5 | Relaxed, aware |
| Beta | 12.5-30 | Alert, attentive |
| Gamma | 30-40 | Focus, concentration |

Across the MAD cohorts, quantitative EEG (qEEG) was recorded for some subjects shortly before administering the final dose (day 7) and about 1 hr, about 2 hr, about 4 hr, about 8 hr, and about 12 hr after administering the final dose. The power in each band recorded shortly before administering the first dose (day 10 provided a baseline (100%) against which the post-dose EEG band power was normalized).

Preliminary data from the MAD1 cohort, in which each dose contained 60 mg of the peptide, indicated pronounced increases in power in the alpha band, from about 80% of baseline before administering the final dose to about 150% of baseline only 1 hr after the final dose. Alpha band power varied from about 120% to about 200% until about 12 hr after the final dose. Gamma band power similarly increased from about 100-110% of baseline before administering the final dose to about 140% of baseline 1 hr after the final dose, and remained above about 125% of baseline for 8 hr.

Preliminary data from the MAD2 cohort, in which each dose contained 180 mg of the peptide, also indicated pronounced increases in power in the alpha and gamma bands. Alpha band power increased from about 110% of baseline before administering the final dose to about 150% of baseline 2 hr after the final dose, and remained above 110% of baseline until about 12 hr after the final dose. Gamma band power increased from about 100-110% of baseline before administering the final dose to about 125% of baseline 1 hr after the final dose, and was above about 150% of baseline after 12 hr.

Preliminary data from the MAD3 cohort, in which each dose contained 540 mg of the peptide, indicated decreases in alpha band power from about 100% of baseline before administering the final dose to about 70-80% of baseline from 1 hr to 12 hr after administering the final dose.

In summary, preliminary data from the study shows that treatment with the peptide at doses of 60 mg/day or 180 mg/day increased the power of alpha and gamma EEG bands, indicative of states of relaxed awareness and focused concentration.

### 8. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.

1. A method of (a) treating a subject suffering from or at risk of an inflammatory disease or disorder, schizophrenia or psychosis, a neurodegenerative disease such as Parkinson's disease, Alzheimer's disease, or ALS, a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, or a sleep disorder such as insomnia, daytime fatigue, or REM sleep behavior disorder, (b) improving sleep in a subject, or (c) improving cognition in a subject, optionally wherein for (a)-(c) the subject has an elevated TNF-α level, comprising administering to the subject an effective amount of a peptide or a pharmaceutically acceptable salt thereof, optionally wherein the peptide or pharmaceutically acceptable salt thereof is administered orally, wherein the peptide:
a. is 5 to 15 amino acids in length; and
b. has an amino acid sequence comprising at least 5 consecutive amino acids from the amino acid sequence LSSTQAQQSX₁ (SEQ ID NO:1), where X₁ is Y, W, or F.

2. The method of embodiment 1, wherein the amino acid sequence of the peptide or pharmaceutically acceptable salt thereof comprises or consists of SSTQA (SEQ ID NO:5).

3. The method of embodiment 1, wherein the amino acid sequence of the peptide or pharmaceutically acceptable salt thereof comprises or consists of STQAQ (SEQ ID NO:6).

4. The method of embodiment 1, wherein the amino acid sequence of the peptide or pharmaceutically acceptable salt thereof comprises or consists of TQAQQ (SEQ ID NO:7).

5. The method of embodiment 1, wherein the amino acid sequence of the peptide or pharmaceutically acceptable salt thereof comprises or consists of QAQQS (SEQ ID NO:8).

6. The method of embodiment 1, wherein the amino acid sequence of the peptide or pharmaceutically acceptable salt thereof comprises or consists of AQQSW (SEQ ID NO:9).

7. The method of embodiment 1, wherein the amino acid sequence of the peptide or pharmaceutically acceptable salt thereof comprises or consists of AQQSF (SEQ ID NO:10).

8. The method of embodiment 1, wherein the amino acid sequence of the peptide or pharmaceutically acceptable salt thereof comprises or consists of LSSTQ (SEQ ID NO:11).

9. The method of embodiment 1, wherein the amino acid sequence of the peptide or pharmaceutically acceptable salt thereof comprises or consists of AQQSY (SEQ ID NO:12).

10. The method of embodiment 1, wherein the amino acid sequence of the peptide or pharmaceutically acceptable salt thereof comprises or consists of LSSTQA (SEQ ID NO:13).

11. The method of embodiment 1, wherein the amino acid sequence of the peptide or pharmaceutically acceptable salt thereof comprises or consists of SSTQAQ (SEQ ID NO:14).

12. The method of embodiment 1, wherein the amino acid sequence of the peptide or pharmaceutically acceptable salt thereof comprises or consists of STQAQQ (SEQ ID NO:15).

13. The method of embodiment 1, wherein the amino acid sequence of the peptide or pharmaceutically acceptable salt thereof comprises or consists of TQAQQS (SEQ ID NO:16).

14. The method of embodiment 1, wherein the amino acid sequence of the peptide or pharmaceutically acceptable salt thereof comprises or consists of QAQQSY (SEQ ID NO: 17).

15. The method of embodiment 1, wherein the amino acid sequence of the peptide or pharmaceutically acceptable salt thereof comprises or consists of QAQQSW (SEQ ID NO: 18).

16. The method of embodiment 1, wherein the amino acid sequence of the peptide or pharmaceutically acceptable salt thereof comprises or consists of QAQQSF (SEQ ID NO:19).

17. The method of embodiment 1, wherein the amino acid sequence of the peptide or pharmaceutically acceptable salt thereof comprises or consists of WLSSTQ (SEQ ID NO:20).

18. The method of embodiment 1, wherein the amino acid sequence of the peptide or pharmaceutically acceptable salt thereof comprises or consists of AQQSYW (SEQ ID NO: 21).

19. The method of embodiment 1, wherein the amino acid sequence of the peptide or pharmaceutically acceptable salt thereof comprises or consists of LSSTQAQ (SEQ ID NO: 22).

20. The method of embodiment 1, wherein the amino acid sequence of the peptide or pharmaceutically acceptable salt thereof comprises or consists of SSTQAQQ (SEQ ID NO: 23).

21. The method of embodiment 1, wherein the amino acid sequence of the peptide or pharmaceutically acceptable salt thereof comprises or consists of STQAQQS (SEQ ID NO: 24).

22. The method of embodiment 1, wherein the amino acid sequence of the peptide or pharmaceutically acceptable salt thereof comprises or consists of TQAQQSY (SEQ ID NO: 25).

23. The method of embodiment 1, wherein the amino acid sequence of the peptide or pharmaceutically acceptable salt thereof comprises or consists of TQAQQSW (SEQ ID NO: 26).

24. The method of embodiment 1, wherein the amino acid sequence of the peptide or pharmaceutically acceptable salt thereof comprises or consists of TQAQQSF (SEQ ID NO: 27).

25. The method of embodiment 1, wherein the amino acid sequence of the peptide or pharmaceutically acceptable salt thereof comprises or consists of LSSTQAQQ (SEQ ID NO: 28).

26. The method of embodiment 1, wherein the amino acid sequence of the peptide or pharmaceutically acceptable salt thereof comprises or consists of SSTQAQQS (SEQ ID NO: 29).

27. The method of embodiment 1, wherein the amino acid sequence of the peptide or pharmaceutically acceptable salt thereof comprises or consists of STQAQQSY (SEQ ID NO: 30).

28. The method of embodiment 1, wherein the amino acid sequence of the peptide or pharmaceutically acceptable salt thereof comprises or consists of STQAQQSW (SEQ ID NO: 31).

29. The method of embodiment 1, wherein the amino acid sequence of the peptide or pharmaceutically acceptable salt thereof comprises or consists of STQAQQSF (SEQ ID NO: 32).

30. The method of embodiment 1, wherein the amino acid sequence of the peptide or pharmaceutically acceptable salt thereof comprises or consists of SSTQAQQSW (SEQ ID NO: 33).

31. The method of embodiment 1, wherein the amino acid sequence of the peptide or pharmaceutically acceptable salt thereof comprises or consists of SSTQAQQSF (SEQ ID NO: 34).

32. The method of embodiment 1, wherein the amino acid sequence of the peptide or pharmaceutically acceptable salt thereof comprises or consists of LSSTQAQQS (SEQ ID NO: 35).

33. The method of embodiment 1, wherein the amino acid sequence of the peptide or pharmaceutically acceptable salt thereof comprises or consists of SSTQAQQSY (SEQ ID NO: 36).

34. The method of embodiment 1, wherein the amino acid sequence of the peptide or pharmaceutically acceptable salt thereof comprises or consists of LSSTQAQQSY (SEQ ID NO: 2).

35. The method of embodiment 1, wherein the amino acid sequence of the peptide or pharmaceutically acceptable salt thereof comprises or consists of LSSTQAQQSW (SEQ ID NO: 3).

36. The method of embodiment 1, wherein the amino acid sequence of the peptide or pharmaceutically acceptable salt thereof comprises or consists of LSSTQAQQSF (SEQ ID NO: 4).

37. The method of embodiment 1, wherein the amino acid sequence of the peptide or pharmaceutically acceptable salt thereof comprises or consists of LSSTQAQQSYW (SEQ ID NO: 38).

38. The method of embodiment 1, wherein the amino acid sequence of the peptide or pharmaceutically acceptable salt thereof comprises or consists of WLSSTQAQQSY (SEQ ID NO: 39).

39. The method of embodiment 1, wherein the amino acid sequence of the peptide or pharmaceutically acceptable salt thereof comprises or consists of WLSSTQAQQSYW (SEQ ID NO:40).

40. The method of any one of embodiments 1 to 9, wherein the peptide is 5 amino acids in length.

41. The method of any one of embodiments 1 to 18, wherein the peptide is 6 amino acids in length.

42. The method of any one of embodiments 1 to 24, wherein the peptide is 7 amino acids in length.

43. The method of any one of embodiments 1 to 29, wherein the peptide is 8 amino acids in length. 44. The method of any one of embodiments 1 to 33, wherein the peptide is 9 amino acids in length.

45. The method of any one of embodiments 1 to 36, wherein the peptide is 10 amino acids in length.

46. The method of any one of embodiments 1 to 38, wherein the peptide is 11 amino acids in length.

47. The method of any one of embodiments 1 to 39, wherein the peptide is 12 amino acids in length.

48. The method of any one of embodiments 1 to 39, wherein the peptide is 13 amino acids in length.

49. The method of any one of embodiments 1 to 39, wherein the peptide is 14 amino acids in length.

50. The method of any one of embodiments 1 to 39, wherein the peptide is 15 amino acids in length.

51. A method of (a) treating a subject suffering from or at risk of an inflammatory disease or disorder, schizophrenia or psychosis, a neurodegenerative disease such as Parkinson's disease, Alzheimer's disease, or ALS, a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, or a sleep disorder such as insomnia, daytime fatigue, or REM sleep behavior disorder, (b) improving sleep in a subject, or (c) improving cognition in a subject, optionally wherein for (a)-(c) the subject has an elevated TNF-α level, comprising administering to the subject an effective amount of a peptide conjugate or a pharmaceutically acceptable salt thereof, optionally wherein the peptide conjugate or pharmaceutically acceptable salt thereof is administered orally, wherein the peptide conjugate comprises a peptide moiety attached to a conjugate moiety, and wherein the peptide moiety:
a. is 5 to 15 amino acids in length; and
b. has an amino acid sequence comprising at least 5 consecutive amino acids from the amino acid sequence LSSTQAQQSX₁ (SEQ ID NO:1), where X₁ is Y, W, or F.

52. The method of embodiment 51, wherein the peptide moiety is a peptide as described in any one of embodiments 2 to 50.

53. The method of embodiment 51 or embodiment 52, wherein at least one of the one or more conjugate moieties comprises a polymer, an amino group, an acyl group, an alkyl group, a phosphate group, a lipid or a sugar.

54. The method of embodiment 53, wherein at least one of the one or more conjugate moieties comprises a polymer.

55. The method of embodiment 54, wherein the polymer comprises a polyethylene glycol, polyvinyl pyrrolidone, polylactic-co-glycolic acid, N-(2-hydroxypropyl) methacrylamide copolymer, polyglutamic acid, or a polysaccharide.

56. The method of any one of embodiments 51 to 55, wherein at least one of the one or more conjugate moieties comprises an amine group.

57. The method of embodiment 56, wherein the amine group is an amino group, an alkyl amino group, or a dialkyl amino group.

58. The method of any one of embodiments 51 to 57, wherein at least one of the one or more conjugate moieties comprises an acyl group.

59. The method of embodiment 58, wherein the acyl group is a formyl group or an acetyl group.

60. The method of any one of embodiments 51 to 59, wherein at least one of the one or more conjugate moieties comprises an alkyl group.

61. The method of embodiment 60, wherein the alkyl group is a methyl group or an ethyl group.

62. The method of any one of embodiments 51 to 61, wherein at least one of the one or more conjugate moieties comprises a phosphate group.

63. The method of any one of embodiments 51 to 62, wherein at least one of the one or more conjugate moieties comprises a lipid.

64. The method of any one of embodiments 51 to 63, wherein at least one of the one or more conjugate moieties comprises a sugar.

65. The method of any one of embodiments 51 to 64, which comprises a single conjugate moiety.

66. The method of embodiment 65, wherein the conjugate moiety is attached to the N-terminal amino acid of the peptide moiety.

67. The method of embodiment 65, wherein the conjugate moiety is attached to the C-terminal amino acid of the peptide moiety.

68. The method of any one of embodiments 51 to 67, which comprises more than one conjugate moiety.

69. The method of embodiment 68, wherein all of the conjugate moieties are the same.

70. The method of embodiment 68, wherein not all of the conjugate moieties are the same.

71. The method of embodiment 68, wherein all of the conjugate moieties are different.

72. The method of embodiment 68, which comprises a conjugate moiety attached to the N-terminal amino acid of the peptide moiety and a conjugate moiety attached to the C-terminal amino acid of the peptide moiety.

73. The method of embodiment 72, wherein the conjugate moiety attached to the N-terminal amino acid of the peptide moiety is the same as the conjugate moiety attached to the C-terminal amino acid of the peptide moiety.

74. The method of embodiment 72, wherein the conjugate moiety attached to the N-terminal amino acid of the peptide moiety is different from the conjugate moiety attached to the C-terminal amino acid of the peptide moiety.

75. The method of any one of embodiments 1 to 74, wherein the subject is suffering from or at risk of schizophrenia or psychosis.

76. The method of embodiment 75, wherein the subject is suffering from schizophrenia.

77. The method of any one of embodiments 75 to 76, wherein the subject exhibits psychotic behavior.

78. The method of any one of embodiments 75 to 77, wherein the treating comprises improving or slowing progression of one or more negative symptoms of schizophrenia or psychosis.

79. The method of embodiment 78, wherein the one or more negative symptoms comprise asociality, anhedonia, alogia, affective flattening, apathy, avolition, blunted affect, anergia, apathy, depression, low mood, cognitive impairment or a combination thereof.

80. The method of embodiment 79, wherein the one or more negative symptoms comprise asociality.

81. The method of embodiment 79 or embodiment 80, wherein the one or more negative symptoms comprise anhedonia.

82. The method of any one of embodiments 79 to 81, wherein the one or more negative symptoms comprise alogia.

83. The method of any one of embodiments 79 to 82, wherein the one or more negative symptoms comprise affective flattening.

84. The method of any one of embodiments 79 to 83, wherein the one or more negative symptoms comprise apathy.

85. The method of any one of embodiments 79 to 84, wherein the one or more negative symptoms comprise avolition.

86. The method of any one of embodiments 79 to 85, wherein the one or more negative symptoms comprise blunted affect.

87. The method of any one of embodiments 79 to 86, wherein the one or more negative symptoms comprise anergia.

88. The method of any one of embodiments 79 to 87, wherein the one or more negative symptoms comprise apathy.

89. The method of any one of embodiments 79 to 88, wherein the one or more negative symptoms comprise depression.

90. The method of any one of embodiments 79 to 89, wherein the one or more negative symptoms comprise low mood.

91. The method of any one of embodiments 79 to 90, wherein the one or more negative symptoms comprise cognitive impairment.

92. The method of any one of embodiments 75 to 91, wherein the treating comprises improving or slowing progression of a cognitive deficit in the subject, optionally wherein the cognitive deficit comprises a deficit in verbal working memory, spatial working memory, verbal fluency, verbal learning, or a combination thereof.

93. The method of any one of embodiments 75 to 92, wherein the treating comprises improving or slowing worsening of the subject's Positive and Negative Syndrome Scale (PANSS) negative symptom factor score.

94. The method of any one of embodiments 75 to 93, wherein the treating comprises improving or slowing worsening of the subject's Scale for the Assessment of Negative Symptoms (SANS) score.

95. The method of any one of embodiments 75 to 94, wherein the treating comprises improving or slowing worsening of the subject's Personal and Social Performance (PSP) total score.

96. The method of any one of embodiments 75 to 95, wherein the treating comprises improving or slowing worsening of the subject's PANSS total score.

97. The method of any one of embodiments 75 to 96, wherein the treating comprises improving or slowing worsening of one or more of the subject's PANSS factor scores.

98. The method of any one of embodiments 75 to 97, wherein the treating comprises improving or slowing worsening of one or more of the subject's PANSS subscale scores.

99. The method of any one of embodiments 75 to 98, wherein the treating comprises improving or slowing worsening of the subject's Clinical Global Impression Improvement (CGI-I) overall score.

100. The method of any one of embodiments 75 to 99, wherein the treating comprises improving or slowing worsening of the subject's Clinical Global Impression Improvement (CGI-I) negative symptoms rating score.

101. The method of any one of embodiments 75 to 100, wherein the treating comprises improving or slowing worsening of the subject's Clinical Global Impression Severity (CGI-S) overall score.

102. The method of any one of embodiments 75 to 101, wherein the treating comprises improving or slowing worsening of the subject's Clinical Global Impression Severity (CGI-S) negative symptoms score.

103. The method of any one of embodiments 1 to 74, wherein the subject is suffering from a neurodegenerative disease.

104. The method of embodiment 103, wherein the subject is suffering from Parkinson's disease.

105. The method of embodiment 103, wherein the subject is suffering from Alzheimer's disease.

106. The method of embodiment 103, wherein the subject is suffering from ALS.

107. The method of any one of embodiments 103 to 106, wherein the treating comprises improving or slowing progression of one or more negative symptoms of the neurodegenerative disease.

108. The method of embodiment 107, wherein the one or more negative symptoms comprise asociality, anhedonia, alogia, affective flattening, apathy, avolition, blunted affect, anergia, apathy, depression, low mood, cognitive impairment or a combination thereof.

109. The method of embodiment 108, wherein the one or more negative symptoms comprise asociality.

110. The method of embodiment 108 or embodiment 109, wherein the one or more negative symptoms comprise anhedonia.

111. The method of any one of embodiments 108 to 110, wherein the one or more negative symptoms comprise alogia.

112. The method of any one of embodiments 108 to 111, wherein the one or more negative symptoms comprise affective flattening.

113. The method of any one of embodiments 108 to 112, wherein the one or more negative symptoms comprise apathy.

114. The method of any one of embodiments 108 to 113, wherein the one or more negative symptoms comprise avolition.

115. The method of any one of embodiments 108 to 114, wherein the one or more negative symptoms comprise blunted affect.

116. The method of any one of embodiments 108 to 115, wherein the one or more negative symptoms comprise anergia.

117. The method of any one of embodiments 108 to 116, wherein the one or more negative symptoms comprise apathy.

118. The method of any one of embodiments 108 to 117, wherein the one or more negative symptoms comprise depression.

119. The method of any one of embodiments 108 to 118, wherein the one or more negative symptoms comprise low mood.

120. The method of any one of embodiments 108 to 119, wherein the one or more negative symptoms comprise cognitive impairment.

121. The method of any one of embodiments 1 to 74, wherein the subject is suffering from Parkinson's disease.

122. The method of embodiment 121, wherein the treating comprises improving or slowing progression of one or more non-motor symptoms of the subject's Parkinson's disease.

123. The method of embodiment 122, wherein the one or more non-motor symptoms comprise a sensory symptom, a cognitive symptom, autonomic symptom, or a combination thereof.

124. The method of embodiment 123, wherein the one or more non-motor symptoms comprise one or more sensory symptoms.

125. The method of embodiment 124, wherein the one or more sensory symptoms comprise numbness, restlessness, pain, chest discomfort, anosmia, or a combination thereof.

126. The method of any one of embodiments 123 to 125, wherein the one or more non-motor symptoms comprise one or more cognitive symptoms.

127. The method of embodiment 126, wherein the one or more cognitive symptoms comprise mood changes, depression, anxiety, panic attacks, tiredness, confusion, slowed thinking, or a combination thereof.

128. The method of any one of embodiments 123 to 127, wherein the one or more non-motor symptoms comprise one or more autonomic symptoms.

129. The method of embodiment 128, wherein the one or more autonomic symptoms comprise hot/cold sensations, bladder problems, sweating, abdominal discomfort, constipation, sialorrhea, frequent urination and/or urgency, erectile dysfunction, or a combination thereof.

130. The method of any one of embodiments 122 to 129, wherein the one or more non-motor symptoms comprise cognition deficit and/or impairment, depression, anxiety, fatigue, apathy, or a combination thereof.

131. The method of any one of embodiments 121 to 130, wherein the treating comprises improving or slowing worsening of the subject's Non-motor Symptoms Scale (NMSS) Score.

132. The method of any one of embodiments 121 to 131, wherein the treating comprises improving or slowing worsening of the subject's MDS-UPDRS non-motor symptoms score.

133. The method of any one of embodiments 121 to 132, wherein the treating comprises improving or slowing worsening of the subject's Fatigue Severity Scale (FSS) score.

134. The method of any one of embodiments 121 to 133, wherein the treating comprises improving or slowing worsening of the subject's Beck's Depression Inventory II (BDI-II) score.

135. The method of any one of embodiments 121 to 134, wherein the treating comprises improving or slowing worsening of the subject's Beck Anxiety Inventory (BAI) score.

136. The method of any one of embodiments 121 to 135, wherein the treating comprises improving or slowing worsening of the subject's Montreal Cognitive Assessment (MoCA) score.

137. The method of any one of embodiments 121 to 136, wherein the treating comprises improving or slowing worsening of the subject's score on one or more Cogstate tests.

138. The method of any one of embodiments 121 to 137, wherein the treating comprises improving or slowing worsening of the subject's Clinical Global Impression Improvement (CGI-I) overall score.

139. The method of any one of embodiments 121 to 138, wherein the treating comprises improving or slowing worsening of the subject's Clinical Global Impression Improvement (CGI-I) non-motor symptoms score.

140. The method of any one of embodiments 121 to 139, wherein the treating comprises improving or slowing worsening of the subject's Clinical Global Impression Severity (CGI-S) overall score.

141. The method of any one of embodiments 121 to 140, wherein the treating comprises improving or slowing worsening of the subject's Clinical Global Impression Severity (CGI-S) non-motor symptoms score.

142. The method of any one of embodiments 121 to 141, wherein the treating comprises improving or slowing worsening of the subject's Parkinson's Disease Questionnaire-39 (PDQ-39) score.

143. The method of any one of embodiments 1 to 74, wherein the subject is suffering from a gastrointestinal disease or disorder.

144. The method of any one of embodiments 1 to 74, wherein the subject is suffering from irritable bowel syndrome.

145. The method of any one of embodiments 1 to 74, wherein the subject is suffering from inflammatory bowel disease.

146. The method of any one of embodiments 1 to 74, wherein the subject is suffering from ulcerative colitis.

147. The method of any one of embodiments 1 to 74, wherein the subject is suffering from Crohn's disease.

148. The method of any one of embodiments 1 to 74, wherein the subject is suffering from constipation.

149. The method of any one of embodiments 1 to 74, wherein the subject is suffering from pain.

150. The method of any one of embodiments 1 to 74, wherein the subject is suffering from visceral pain.

151. The method of any one of embodiments 1 to 74, wherein the subject is suffering from rheumatoid arthritis.

152. The method of any one of embodiments 1 to 74, wherein the subject is suffering from migraine.

153. The method of any one of embodiments 1 to 74, wherein the subject is suffering from headache.

154. The method of any one of embodiments 1 to 74, wherein the subject is suffering from substance abuse.

155. The method of any one of embodiments 1 to 74, wherein the subject is suffering from a substance use disorder, optionally wherein the substance use disorder is drug use disorder, polysubstance use disorder, alcohol use disorder, nicotine use disorder, or tobacco use disorder.

156. The method of any one of embodiments 1 to 74, wherein the subject is suffering from drug addiction.

157. The method of embodiment 156, wherein the subject is suffering from an opioid addiction (e.g., morphine, heroin, oxycodone, or fentanyl).

158. The method of embodiment 156, wherein the subject is suffering from a cocaine addiction.

159. The method of embodiment 156, wherein the subject is suffering from a benzodiazepine addiction (e.g., diazepam, alprazolam, or clonazepam).

160. The method of any one of embodiments 1 to 74, wherein the subject is suffering from a seizure disorder.

161. The method of embodiment 160, wherein the seizure disorder is epilepsy.

162. The method of embodiment 161, wherein the epilepsy is an orphan epilepsy.

163. The method of any one of embodiments 1 to 74, wherein the subject is suffering from major depressive disorder.

164. The method of any one of embodiments 1 to 74, wherein the subject is suffering from atypical depression.

165. The method of any one of embodiments 1 to 74, wherein the subject is suffering from a major depressive episode (MDE) (e.g., atypical MDE).

166. The method of any one of embodiments 1 to 74, wherein the subject is suffering from treatment resistant depression.

167. The method of any one of embodiments 1 to 74, wherein the subject is suffering from depression in the presence of a neurodegenerative disease (e.g., Parkinson's disease or Alzheimer's disease).

168. The method of embodiment 167, wherein the subject is suffering from depression in the presence of Parkinson's disease.

169. The method of embodiment 167, wherein the subject is suffering from depression in the presence of Alzheimer's disease.

170. The method of embodiment 167, wherein the subject is suffering from depression in the presence of ALS.

171. The method of any one of embodiments 1 to 74, wherein the subject is suffering from cognitive impairment.

172. The method of embodiment 171, wherein the cognitive impairment is minor cognitive impairment.

173. The method of any one of embodiments 1 to 74, wherein the subject is suffering from COVID-19 related cognitive impairment and/or depression.

174. The method of any one of embodiments 1 to 74, wherein the subject is suffering from Alzheimer's disease.

175. The method of any one of embodiments 1 to 74, wherein the subject is suffering from ADHD.

176. The method of any one of embodiments 1 to 74, wherein the subject is suffering from an autism spectrum disorder.

177. The method of any one of embodiments 1 to 74, wherein the subject is suffering from a pervasive developmental disorder, which is optionally Asperger syndrome or Rett syndrome.

178. The method of any one of embodiments 1 to 74, wherein the subject is suffering from atypical autism.

179. The method of any one of embodiments 1 to 74, wherein the subject is suffering from multiple sclerosis.

180. The method of any one of embodiments 1 to 74, wherein the subject is suffering from PTSD.

181. The method of any one of embodiments 1 to 74, wherein the subject is suffering from a sleep disorder.

182. The method of any one of embodiments 1 to 74, wherein the subject is suffering from insomnia.

183. The method of any one of embodiments 1 to 74, wherein the subject is suffering from daytime fatigue.

184. The method of any one of embodiments 1 to 74, wherein the subject is suffering from REM sleep behavior disorder.

185. The method of any one of embodiments 1 to 74, wherein the treating comprises improving sleep in a subject.

186. The method of any one of embodiments 1 to 185, wherein the subject has an elevated TNF-α level.

187. The method of any one of embodiments 1 to 186, wherein the treating comprises improving or slowing worsening of the subject's overall Patient Health Questionnaire-9 (PHQ-9) score, optionally wherein the subject does not suffer from a condition set forth in (a) or is a healthy subject.

188. The method of any one of embodiments 1 to 187, wherein the treating comprises improving or slowing worsening of the subject's score on one or more of questions of PHQ-9, optionally wherein the subject does not suffer from a condition set forth in (a) or is a healthy subject.

189. The method of embodiment 188, wherein the treating comprises improving or slowing worsening of the subject's score on one or more of PHQ-9 questions 1, 2, 3, or 6.

190. The method of any one of embodiments 1 to 189, wherein the treating comprises improving or slowing worsening of the subject's score in one or more of the CogState Groton Maze Learning Test (GMLT), the CogState Identification Test (IDN), or the CogState One Card Learning Test (OCL), optionally wherein the subject does not suffer from a condition set forth in (a) or is a healthy subject.

191. The method of any one of embodiments 1 to 190, wherein the treating comprises improving or slowing worsening of at least one of the alpha band power or the gamma band power of the subject's electroencephalogram (EEG), optionally wherein the subject does not suffer from a condition set forth in (a) or is a healthy subject.

192. The method of any one of embodiments 1 to 191, wherein the peptide or pharmaceutically acceptable salt thereof, or the peptide conjugate or pharmaceutically acceptable salt thereof is administered at a dose from about 50 mg/day to about 750 mg/day or about 60 mg/day to about 540 mg/day, optionally at a dose of 60 mg/day, 180 mg/day, or 540 mg/day.

193. The method of any one of embodiments 1 to 192, wherein the peptide or pharmaceutically acceptable salt thereof, or the peptide conjugate or pharmaceutically acceptable salt thereof is administered from once over a period of about four days to about seven times over a period of about ten days.

194. The method of any one of embodiments 1 to 192, wherein about 60 mg/day of the peptide or pharmaceutically acceptable salt thereof, or the peptide conjugate or pharmaceutically acceptable salt thereof is administered once over a period of about four days.

195. The method of any one of embodiments 1 to 192, wherein about 180 mg/day of the peptide or pharmaceutically acceptable salt thereof, or the peptide conjugate or pharmaceutically acceptable salt thereof is administered once over a period of about four days.

196. The method of any one of embodiments 1 to 192, wherein about 540 mg/day of the peptide or pharmaceutically acceptable salt thereof, or the peptide conjugate or pharmaceutically acceptable salt thereof is administered once over a period of about four days.

197. The method of any one of embodiments 1 to 192, wherein about 60 mg/day of the peptide or pharmaceutically acceptable salt thereof, or the peptide conjugate or pharmaceutically acceptable salt thereof is administered about seven times over a period of about ten days.

198. The method of any one of embodiments 1 to 192, wherein about 180 mg/day of the peptide or pharmaceutically acceptable salt thereof, or the peptide conjugate or pharmaceutically acceptable salt thereof is administered about seven times over a period of about ten days.

199. The method of any one of embodiments 1 to 192, wherein about 540 mg/day of the peptide or pharmaceutically acceptable salt thereof, or the peptide conjugate or pharmaceutically acceptable salt thereof is administered about seven times over a period of about ten days.

200. A peptide or a pharmaceutically acceptable salt thereof for use in a method of (a) treating a subject suffering from or at risk of an inflammatory disease or disorder, schizophrenia or psychosis, a neurodegenerative disease such as Parkinson's disease, Alzheimer's disease, or ALS, a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, a major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder. (b) improving sleep in a subject, or (c) improving cognition in a subject, optionally wherein for (a)-(c) the subject has an elevated TNF-α level, wherein the peptide:
a. is 5 to 15 amino acids in length; and
b. has an amino acid sequence comprising at least 5 consecutive amino acids from the amino acid sequence LSSTQAQQSX; (SEQ ID NO:1), where X₁ is Y, W, or F,
   optionally wherein the method comprises administering the peptide or pharmaceutically acceptable salt thereof orally.

201. The peptide or a pharmaceutically acceptable salt thereof for use according to embodiment 200, wherein the amino acid sequence of the peptide or pharmaceutically acceptable salt thereof is LSSTQAQQSY (SEQ ID NO:2), LSSTQAQQSW (SEQ ID NO:3), or LSSTQAQQSF (SEQ ID NO:4).

202. The peptide or a pharmaceutically acceptable salt thereof for use according to embodiment 201, wherein the amino acid sequence of the peptide or pharmaceutically acceptable salt thereof is LSSTQAQQSY (SEQ ID NO:2).

203. The peptide or a pharmaceutically acceptable salt thereof for use according to embodiment 200, wherein the peptide is a peptide as described in any one of embodiments 2 to 50.

204. A peptide conjugate or a pharmaceutically acceptable salt thereof for use in a method of (a) treating a subject suffering from or at risk of an inflammatory disease or disorder, schizophrenia or psychosis, a neurodegenerative disease such as Parkinson's disease, Alzheimer's disease, or ALS, a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, a major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder, (b) improving sleep in a subject, or (c) improving cognition in a subject, optionally wherein for (a)-(c) the subject has an elevated TNF-α level, wherein the peptide conjugate comprises a peptide moiety attached to a conjugate moiety, wherein the peptide moiety:
a. is 5 to 15 amino acids in length; and
b. has an amino acid sequence comprising at least 5 consecutive amino acids from the amino acid sequence LSSTQAQQSX₁ (SEQ ID NO:1), where X₁ is Y, W, or F,
   optionally wherein the method comprises administering the peptide conjugate or pharmaceutically acceptable salt thereof orally.

205. The peptide conjugate or a pharmaceutically acceptable salt thereof for use according to embodiment 204, wherein the peptide moiety is a peptide as described in any one of embodiments 2 to 50.

206. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from or at risk of schizophrenia or psychosis.

207. The peptide or pharmaceutically acceptable salt thereof or peptide conjugate or pharmaceutically acceptable salt thereof for use according to embodiment 206, wherein the subject is suffering from schizophrenia.

208. The peptide or pharmaceutically acceptable salt thereof or peptide conjugate or pharmaceutically acceptable salt thereof for use according to embodiment 206 or embodiment 207, wherein the subject exhibits psychotic behavior.

209. The peptide or pharmaceutically acceptable salt thereof or peptide conjugate or pharmaceutically acceptable salt thereof for use according to any one of embodiments 206 to 208, wherein the treating comprises improving or slowing progression of one or more negative symptoms of schizophrenia or psychosis.

210. The peptide or pharmaceutically acceptable salt thereof or peptide conjugate or pharmaceutically acceptable salt thereof for use according to embodiment 209, wherein the one or more negative symptoms comprise asociality, anhedonia, alogia, affective flattening, apathy, avolition, blunted affect, anergia, apathy, depression, low mood, cognitive impairment or a combination thereof.

211. The peptide or pharmaceutically acceptable salt thereof or peptide conjugate or pharmaceutically acceptable salt thereof for use according to embodiment 210, wherein the one or more negative symptoms comprise asociality.

212. The peptide or pharmaceutically acceptable salt thereof or peptide conjugate or pharmaceutically acceptable salt thereof for use according to any one of embodiments 210 to 211, wherein the one or more negative symptoms comprise anhedonia.

213. The peptide or pharmaceutically acceptable salt thereof or peptide conjugate or pharmaceutically acceptable salt thereof for use according to any one of embodiments 210 to 212, wherein the one or more negative symptoms comprise alogia.

214. The peptide or pharmaceutically acceptable salt thereof or peptide conjugate or pharmaceutically acceptable salt thereof for use according to any one of embodiments 210 to 213, wherein the one or more negative symptoms comprise affective flattening.

215. The peptide or pharmaceutically acceptable salt thereof or peptide conjugate or pharmaceutically acceptable salt thereof for use according to any one of embodiments 210 to 214, wherein the one or more negative symptoms comprise apathy.

216. The peptide or pharmaceutically acceptable salt thereof or peptide conjugate or pharmaceutically acceptable salt thereof for use according to any one of embodiments 210 to 215, wherein the one or more negative symptoms comprise avolition.

217. The peptide or pharmaceutically acceptable salt thereof or peptide conjugate or pharmaceutically acceptable salt thereof for use according to any one of embodiments 210 to 216, wherein the one or more negative symptoms comprise blunted affect.

218. The peptide or pharmaceutically acceptable salt thereof or peptide conjugate or pharmaceutically acceptable salt thereof for use according to any one of embodiments 210 to 217, wherein the one or more negative symptoms comprise anergia.

219. The peptide or pharmaceutically acceptable salt thereof or peptide conjugate or pharmaceutically acceptable salt thereof for use according to any one of embodiments 210 to 218, wherein the one or more negative symptoms comprise apathy.

220. The peptide or pharmaceutically acceptable salt thereof or peptide conjugate or pharmaceutically acceptable salt thereof for use according to any one of embodiments 210 to 219, wherein the one or more negative symptoms comprise depression.

221. The peptide or pharmaceutically acceptable salt thereof or peptide conjugate or pharmaceutically acceptable salt thereof for use according to any one of embodiments 210 to 220, wherein the one or more negative symptoms comprise low mood.

222. The peptide or pharmaceutically acceptable salt thereof or peptide conjugate or pharmaceutically acceptable salt thereof for use according to any one of embodiments 210 to 221, wherein the one or more negative symptoms comprise cognitive impairment.

223. The peptide or pharmaceutically acceptable salt thereof or peptide conjugate or pharmaceutically acceptable salt thereof for use according to any one of embodiments 206 to 222, wherein the treating comprises improving or slowing progression of a cognitive deficit in the subject, optionally wherein the cognitive deficit comprises a deficit in verbal working memory, spatial working memory, verbal fluency, verbal learning, or a combination thereof.

224. The peptide or pharmaceutically acceptable salt thereof or peptide conjugate or pharmaceutically acceptable salt thereof for use according to any one of embodiments 206 to 223, wherein the treating comprises improving or slowing worsening of the subject's Positive and Negative Syndrome Scale (PANSS) negative symptom factor score.

225. The peptide or pharmaceutically acceptable salt thereof or peptide conjugate or pharmaceutically acceptable salt thereof for use according to any one of embodiments 206 to 224, wherein the treating comprises improving or slowing worsening of the subject's Scale for the Assessment of Negative Symptoms (SANS) score.

226. The peptide or pharmaceutically acceptable salt thereof or peptide conjugate or pharmaceutically acceptable salt thereof for use according to any one of embodiments 206 to 225, wherein the treating comprises improving or slowing worsening of the subject's Personal and Social Performance (PSP) total score.

227. The peptide or pharmaceutically acceptable salt thereof or peptide conjugate or pharmaceutically acceptable salt thereof for use according to any one of embodiments 206 to 226, wherein the treating comprises improving or slowing worsening of the subject's PANSS total score.

228. The peptide or pharmaceutically acceptable salt thereof or peptide conjugate or pharmaceutically acceptable salt thereof for use according to any one of embodiments 206 to 227, wherein the treating comprises improving or slowing worsening of one or more of the subject's PANSS factor scores.

229. The peptide or pharmaceutically acceptable salt thereof or peptide conjugate or pharmaceutically acceptable salt thereof for use according to any one of embodiments 206 to 228, wherein the treating comprises improving or slowing worsening of one or more of the subject's PANSS subscale scores.

230. The peptide or pharmaceutically acceptable salt thereof or peptide conjugate or pharmaceutically acceptable salt thereof for use according to any one of embodiments 206 to 229, wherein the treating comprises improving or slowing worsening of the subject's Clinical Global Impression Improvement (CGI-I) overall score.

231. The peptide or pharmaceutically acceptable salt thereof or peptide conjugate or pharmaceutically acceptable salt thereof for use according to any one of embodiments 206 to 230, wherein the treating comprises improving or slowing worsening of the subject's Clinical Global Impression Improvement (CGI-I) negative symptoms rating score.

232. The peptide or pharmaceutically acceptable salt thereof or peptide conjugate or pharmaceutically acceptable salt thereof for use according to any one of embodiments 206 to 231, wherein the treating comprises improving or slowing worsening of the subject's Clinical Global Impression Severity (CGI-S) overall score.

233. The peptide or pharmaceutically acceptable salt thereof or peptide conjugate or pharmaceutically acceptable salt thereof for use according to any one of embodiments 206 to 232, wherein the treating comprises improving or slowing worsening of the subject's Clinical Global Impression Severity (CGI-S) negative symptoms score.

234. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a neurodegenerative disease.

235. The peptide or pharmaceutically acceptable salt thereof or peptide conjugate or pharmaceutically acceptable salt thereof for use according to embodiment 234, wherein the subject is suffering from Parkinson's disease.

236. The peptide or pharmaceutically acceptable salt thereof or peptide conjugate or pharmaceutically acceptable salt thereof for use according to embodiment 234, wherein the subject is suffering from Alzheimer's disease.

237. The peptide or pharmaceutically acceptable salt thereof or peptide conjugate or pharmaceutically acceptable salt thereof for use according to embodiment 234, wherein the subject is suffering from ALS.

238. The peptide or pharmaceutically acceptable salt thereof or peptide conjugate or pharmaceutically acceptable salt thereof for use according to any one of embodiments 234 to 237, wherein the treating comprises improving or slowing progression of one or more negative symptoms of the neurodegenerative disease.

239. The peptide or pharmaceutically acceptable salt thereof or peptide conjugate or pharmaceutically acceptable salt thereof for use according to embodiment 238, wherein the one or more negative symptoms comprise asociality, anhedonia, alogia, affective flattening, apathy, avolition, blunted affect, anergia, apathy, depression, low mood, cognitive impairment or a combination thereof.

240. The peptide or pharmaceutically acceptable salt thereof or peptide conjugate or pharmaceutically acceptable salt thereof for use according to embodiment 239, wherein the one or more negative symptoms comprise asociality.

241. The peptide or pharmaceutically acceptable salt thereof or peptide conjugate or pharmaceutically acceptable salt thereof for use according to embodiment 239 or 240, wherein the one or more negative symptoms comprise anhedonia.

242. The peptide or pharmaceutically acceptable salt thereof or peptide conjugate or pharmaceutically acceptable salt thereof for use according to any one of embodiments 239 to 241, wherein the one or more negative symptoms comprise alogia.

243. The peptide or pharmaceutically acceptable salt thereof or peptide conjugate or pharmaceutically acceptable salt thereof for use according to any one of embodiments 239 to 242, wherein the one or more negative symptoms comprise affective flattening.

244. The peptide or pharmaceutically acceptable salt thereof or peptide conjugate or pharmaceutically acceptable salt thereof for use according to any one of embodiments 239 to 243, wherein the one or more negative symptoms comprise apathy.

245. The peptide or pharmaceutically acceptable salt thereof or peptide conjugate or pharmaceutically acceptable salt thereof for use according to any one of embodiments 239 to 244, wherein the one or more negative symptoms comprise avolition.

246. The peptide or pharmaceutically acceptable salt thereof or peptide conjugate or pharmaceutically acceptable salt thereof for use according to any one of embodiments 239 to 245, wherein the one or more negative symptoms comprise blunted affect.

247. The peptide or pharmaceutically acceptable salt thereof or peptide conjugate or pharmaceutically acceptable salt thereof for use according to any one of embodiments 239 to 246, wherein the one or more negative symptoms comprise anergia.

248. The peptide or pharmaceutically acceptable salt thereof or peptide conjugate or pharmaceutically acceptable salt thereof for use according to any one of embodiments 239 to 247, wherein the one or more negative symptoms comprise apathy.

249. The peptide or pharmaceutically acceptable salt thereof or peptide conjugate or pharmaceutically acceptable salt thereof for use according to any one of embodiments 239 to 248, wherein the one or more negative symptoms comprise depression.

250. The peptide or pharmaceutically acceptable salt thereof or peptide conjugate or pharmaceutically acceptable salt thereof for use according to any one of embodiments 239 to 249, wherein the one or more negative symptoms comprise low mood.

251. The peptide or pharmaceutically acceptable salt thereof or peptide conjugate or pharmaceutically acceptable salt thereof for use according to any one of embodiments 239 to 250, wherein the one or more negative symptoms comprise cognitive impairment.

252. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from Parkinson's disease.

253. The peptide or pharmaceutically acceptable salt thereof or peptide conjugate or pharmaceutically acceptable salt thereof for use according to embodiment 252, wherein the treating comprises improving or slowing progression of one or more non-motor symptoms of the subject's Parkinson's disease.

254. The peptide or pharmaceutically acceptable salt thereof or peptide conjugate or pharmaceutically acceptable salt thereof for use according to embodiment 253, wherein the one or more non-motor symptoms comprise a sensory symptom, a cognitive symptom, autonomic symptom, or a combination thereof.

255. The peptide or pharmaceutically acceptable salt thereof or peptide conjugate or pharmaceutically acceptable salt thereof for use according to embodiment 254, wherein the one or more non-motor symptoms comprise one or more sensory symptoms.

256. The peptide or pharmaceutically acceptable salt thereof or peptide conjugate or pharmaceutically acceptable salt thereof for use according to embodiment 255, wherein the one or more sensory symptoms comprise numbness, restlessness, pain, chest discomfort, anosmia, or a combination thereof.

257. The peptide or pharmaceutically acceptable salt thereof or peptide conjugate or pharmaceutically acceptable salt thereof for use according to any one of embodiments 254 to 256, wherein the one or more non-motor symptoms comprise one or more cognitive symptoms.

258. The peptide or pharmaceutically acceptable salt thereof or peptide conjugate or pharmaceutically acceptable salt thereof for use according to embodiment 257, wherein the one or more cognitive symptoms comprise mood changes, depression, anxiety, panic attacks, tiredness, confusion, slowed thinking, or a combination thereof.

259. The peptide or pharmaceutically acceptable salt thereof or peptide conjugate or pharmaceutically acceptable salt thereof for use according to any one of embodiments 254 to 258, wherein the one or more non-motor symptoms comprise one or more autonomic symptoms.

260. The peptide or pharmaceutically acceptable salt thereof or peptide conjugate or pharmaceutically acceptable salt thereof for use according to embodiment 259, wherein the one or more autonomic symptoms comprise hot/cold sensations, bladder problems, sweating, abdominal discomfort, constipation, sialorrhea, frequent urination and/or urgency, erectile dysfunction, or a combination thereof.

261. The peptide or pharmaceutically acceptable salt thereof or peptide conjugate or pharmaceutically acceptable salt thereof for use according to any one of embodiments 253 to 260, wherein the one or more non-motor symptoms comprise cognition deficit and/or impairment, depression, anxiety, fatigue, apathy, or a combination thereof.

262. The peptide or pharmaceutically acceptable salt thereof or peptide conjugate or pharmaceutically acceptable salt thereof for use according to any one of embodiments 252 to 261, wherein the treating comprises improving or slowing worsening of the subject's Non-motor Symptoms Scale (NMSS) Score.

263. The peptide or pharmaceutically acceptable salt thereof or peptide conjugate or pharmaceutically acceptable salt thereof for use according to any one of embodiments 252 to 262, wherein the treating comprises improving or slowing worsening of the subject's MDS-UPDRS non-motor symptoms score.

264. The peptide or pharmaceutically acceptable salt thereof or peptide conjugate or pharmaceutically acceptable salt thereof for use according to any one of embodiments 252 to 263, wherein the treating comprises improving or slowing worsening of the subject's Fatigue Severity Scale (FSS) score.

265. The peptide or pharmaceutically acceptable salt thereof or peptide conjugate or pharmaceutically acceptable salt thereof for use according to any one of embodiments 252 to 264, wherein the treating comprises improving or slowing worsening of the subject's Beck's Depression Inventory II (BDI-II) score.

266. The peptide or pharmaceutically acceptable salt thereof or peptide conjugate or pharmaceutically acceptable salt thereof for use according to any one of embodiments 252 to 265, wherein the treating comprises improving or slowing worsening of the subject's Beck Anxiety Inventory (BAI) score.

267. The peptide or pharmaceutically acceptable salt thereof or peptide conjugate or pharmaceutically acceptable salt thereof for use according to any one of embodiments 252 to 266, wherein the treating comprises improving or slowing worsening of the subject's Montreal Cognitive Assessment (MoCA) score.

268. The peptide or pharmaceutically acceptable salt thereof or peptide conjugate or pharmaceutically acceptable salt thereof for use according to any one of embodiments 252 to 267, wherein the treating comprises improving or slowing worsening of the subject's score on one or more Cogstate tests.

269. The peptide or pharmaceutically acceptable salt thereof or peptide conjugate or pharmaceutically acceptable salt thereof for use according to any one of embodiments 252 to 268, wherein the treating comprises improving or slowing worsening of the subject's Clinical Global Impression Improvement (CGI-I) overall score.

270. The peptide or pharmaceutically acceptable salt thereof or peptide conjugate or pharmaceutically acceptable salt thereof for use according to any one of embodiments 252 to 269, wherein the treating comprises improving or slowing worsening of the subject's Clinical Global Impression Improvement (CGI-I) non-motor symptoms score.

271. The peptide or pharmaceutically acceptable salt thereof or peptide conjugate or pharmaceutically acceptable salt thereof for use according to any one of embodiments 252 to 270, wherein the treating comprises improving or slowing worsening of the subject's Clinical Global Impression Severity (CGI-S) overall score.

272. The peptide or pharmaceutically acceptable salt thereof or peptide conjugate or pharmaceutically acceptable salt thereof for use according to any one of embodiments 252 to 271, wherein the treating comprises improving or slowing worsening of the subject's Clinical Global Impression Severity (CGI-S) non-motor symptoms score.

273. The peptide or pharmaceutically acceptable salt thereof or peptide conjugate or pharmaceutically acceptable salt thereof for use according to any one of embodiments 252 to 272, wherein the treating comprises improving or slowing worsening of the subject's Parkinson's Disease Questionnaire-39 (PDQ-39) score.

274. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a gastrointestinal disease or disorder.

275. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from irritable bowel syndrome.

276. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from inflammatory bowel disease.

277. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from ulcerative colitis.

278. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from Crohn's disease.

279. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from constipation.

280. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from pain.

281. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from visceral pain.

282. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from rheumatoid arthritis.

283. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from migraine.

284. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from headache.

285. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from substance abuse.

286. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a substance use disorder, optionally wherein the substance use disorder is drug use disorder, polysubstance use disorder, alcohol use disorder, nicotine use disorder, or tobacco use disorder.

287. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from drug addiction.

288. The peptide or pharmaceutically acceptable salt thereof for use according to embodiment 287, wherein the subject is suffering from an opioid addiction (e.g., morphine, heroin, oxycodone, or fentanyl).

289. The peptide or pharmaceutically acceptable salt thereof for use according to embodiment 287, wherein the subject is suffering from a cocaine addiction.

290. The peptide or pharmaceutically acceptable salt thereof for use according to embodiment 287, wherein the subject is suffering from a benzodiazepine addiction (e.g., diazepam, alprazolam, or clonazepam).

291. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a seizure disorder.

292. The peptide or pharmaceutically acceptable salt thereof or peptide conjugate or pharmaceutically acceptable salt thereof for use according to embodiment 291, wherein the seizure disorder is epilepsy.

293. The peptide or pharmaceutically acceptable salt thereof or peptide conjugate or pharmaceutically acceptable salt thereof for use according to embodiment 292, wherein the epilepsy is an orphan epilepsy.

294. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from major depressive disorder.

295. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from atypical depression.

296. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a major depressive episode (MDE) (e.g., atypical MDE).

297. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from treatment resistant depression.

298. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from depression in the presence of a neurodegenerative disease (e.g., Parkinson's disease or Alzheimer's disease).

299. The peptide or pharmaceutically acceptable salt thereof or peptide conjugate or pharmaceutically acceptable salt thereof for use according to embodiment 298, wherein the subject is suffering from depression in the presence of Parkinson's disease.

300. The peptide or pharmaceutically acceptable salt thereof or peptide conjugate or pharmaceutically acceptable salt thereof for use according to embodiment 298, wherein the subject is suffering from depression in the presence of Alzheimer's disease.

301. The peptide or pharmaceutically acceptable salt thereof or peptide conjugate or pharmaceutically acceptable salt thereof for use according to embodiment 298, wherein the subject is suffering from depression in the presence of ALS.

302. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from cognitive impairment.

303. The peptide or pharmaceutically acceptable salt thereof or peptide conjugate or pharmaceutically acceptable salt thereof for use according to embodiment 302, wherein the cognitive impairment is minor cognitive impairment.

304. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from COVID-19 related cognitive impairment and/or depression.

305. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from Alzheimer's disease.

306. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from ADHD.

307. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from an autism spectrum disorder.

308. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a pervasive developmental disorder, which is optionally Asperger syndrome or Rett syndrome.

309. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from atypical autism.

310. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from multiple sclerosis.

311. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from PTSD.

312. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from a sleep disorder.

313. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from insomnia.

314. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from daytime fatigue.

315. The peptide or pharmaceutically acceptable salt thereof for use according to any one of embodiments 200 to 203 or the peptide conjugate or pharmaceutically acceptable salt thereof for use according to any one of embodiments 204 to 205, wherein the subject is suffering from REM sleep behavior disorder.

316. The peptide or pharmaceutically acceptable salt thereof for use according to any one of embodiments 200 to 203 or the peptide conjugate or pharmaceutically acceptable salt thereof for use according to any one of embodiments 204 to 205, wherein the treating comprises improving sleep in a subject.

317. The peptide or pharmaceutically acceptable salt thereof or peptide conjugate or pharmaceutically acceptable salt thereof for use according to any one of embodiments 200 to 316, wherein the subject has an elevated TNF-α level.

318. The peptide or pharmaceutically acceptable salt thereof for use according to any one of embodiments 200 to 203 or the peptide conjugate or pharmaceutically acceptable salt thereof for use according to any one of embodiments 204 to 205, wherein the treating comprises improving or slowing worsening of the subject's overall Patient Health Questionnaire-9 (PHQ-9) score, optionally wherein the subject does not suffer from a condition set forth in (a) or is a healthy subject.

319. The peptide or pharmaceutically acceptable salt thereof, or the peptide conjugate or pharmaceutically acceptable salt thereof, for use according to embodiment 318, wherein the treating comprises improving or slowing worsening of the subject's score on one or more of questions of PHQ-9.

320. The peptide or pharmaceutically acceptable salt thereof, or the peptide conjugate or pharmaceutically acceptable salt thereof, for use according to embodiment 319, wherein the treating comprises improving or slowing worsening of the subject's score on one or more of PHQ-9 questions 1, 2, 3, or 6.

321. The peptide or pharmaceutically acceptable salt thereof for use according to any one of embodiments 200 to 203 or the peptide conjugate or pharmaceutically acceptable salt thereof for use according to any one of embodiments 204 to 205, wherein the treating comprises improving or slowing worsening of the subject's score in one or more of the CogState Groton Maze Learning Test (GMLT), the CogState Identification Test (IDN), or the CogState One Card Learning Test (OCL), optionally wherein the subject does not suffer from a condition set forth in (a) or is a healthy subject.

322. The peptide or pharmaceutically acceptable salt thereof for use according to any one of embodiments 200 to 203 or the peptide conjugate or pharmaceutically acceptable salt thereof for use according to any one of embodiments 204 to 205, wherein the treating comprises improving or slowing worsening of at least one of the alpha band power or the gamma band power of the subject's electroencephalogram (EEG), optionally wherein the subject does not suffer from a condition set forth in (a) or is a healthy subject.

323. The peptide or pharmaceutically acceptable salt thereof, or the peptide conjugate or pharmaceutically acceptable salt thereof, for use according to any one of embodiments 200 to 322, wherein the peptide or the pharmaceutically acceptable salt thereof, or the peptide conjugate or pharmaceutically acceptable salt thereof, is provided at a dose from about 50 mg/day to about 750 mg/day or from about 60 mg/day to about 540 mg/day, optionally 60 mg/day. 180 mg/day, or 540 mg/day.

324. The peptide or pharmaceutically acceptable salt thereof, or the peptide conjugate or pharmaceutically acceptable salt thereof, for use according to any one of embodiments 200 to 323, wherein the peptide or pharmaceutically acceptable salt thereof, or the peptide conjugate or pharmaceutically acceptable salt thereof is provided for use from once over a period of about four days to about seven times over a period of about ten days.

325. The peptide or pharmaceutically acceptable salt thereof, or the peptide conjugate or pharmaceutically acceptable salt thereof, for use according to any one of embodiments 200 to 323, wherein about 60 mg/day of the peptide or pharmaceutically acceptable salt thereof, or the peptide conjugate or pharmaceutically acceptable salt thereof is provided for use once over a period of about four days.

326. The peptide or pharmaceutically acceptable salt thereof, or the peptide conjugate or pharmaceutically acceptable salt thereof, for use according to any one of embodiments 200 to 323, wherein about 180 mg/day of the peptide or pharmaceutically acceptable salt thereof, or the peptide conjugate or pharmaceutically acceptable salt thereof is provided for use once over a period of about four days.

327. The peptide or pharmaceutically acceptable salt thereof, or the peptide conjugate or pharmaceutically acceptable salt thereof, for use according to any one of embodiments 200 to 323, wherein about 540 mg/day of the peptide or pharmaceutically acceptable salt thereof, or the peptide conjugate or pharmaceutically acceptable salt thereof is provided for use once over a period of about four days.

328. The peptide or pharmaceutically acceptable salt thereof, or the peptide conjugate or pharmaceutically acceptable salt thereof, for use according to any one of embodiments 200 to 323, wherein about 60 mg/day of the peptide or pharmaceutically acceptable salt thereof, or the peptide conjugate or pharmaceutically acceptable salt thereof is provided for use about seven times over a period of about ten days.

329. The peptide or pharmaceutically acceptable salt thereof, or the peptide conjugate or pharmaceutically acceptable salt thereof, for use according to any one of embodiments 200 to 323, wherein about 180 mg/day of the peptide or pharmaceutically acceptable salt thereof, or the peptide conjugate or pharmaceutically acceptable salt thereof is provided for use about seven times over a period of about ten days.

330. The peptide or pharmaceutically acceptable salt thereof, or the peptide conjugate or pharmaceutically acceptable salt thereof, for use according to any one of embodiments 200 to 323, wherein about 540 mg/day of the peptide or pharmaceutically acceptable salt thereof, or the peptide conjugate or pharmaceutically acceptable salt thereof is provided for use about seven times over a period of about ten days.

331. Use of a peptide or a pharmaceutically acceptable salt thereof for manufacturing a medicament for (a) the treatment or prevention of an inflammatory disease or disorder, schizophrenia or psychosis, a neurodegenerative disease such as Parkinson's disease. Alzheimer's disease, or ALS, a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, a major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder, for (b) improving sleep, or for (c) improving cognition in a subject, optionally wherein for (a)-(c) the medicament is formulated for administration to a subject having an elevated TNF-α level, wherein the peptide:
a. is 5 to 15 amino acids in length; and
b. has an amino acid sequence comprising at least 5 consecutive amino acids from the amino acid sequence LSSTQAQQSX; (SEQ ID NO:1), where X₁ is Y, W, or F,
   optionally wherein medicament is formulated for oral administration.

332. The use of embodiment 331, wherein the amino acid sequence of the peptide or pharmaceutically acceptable salt thereof is LSSTQAQQSY (SEQ ID NO:2), LSSTQAQQSW (SEQ ID NO:3), or LSSTQAQQSF (SEQ ID NO:4).

333. The use of embodiment 332, wherein the amino acid sequence of the peptide or pharmaceutically acceptable salt thereof is LSSTQAQQSY (SEQ ID NO:2).

334. The use of embodiment 331, wherein the peptide is a peptide as described in any one of embodiments 2 to 50.

335. Use of a peptide conjugate or a pharmaceutically acceptable salt thereof for manufacturing a medicament for (a) the treatment or prevention of an inflammatory disease or disorder, schizophrenia or psychosis, a neurodegenerative disease such as Parkinson's disease, Alzheimer's disease, or ALS, a gastrointestinal disease or disorder such as irritable bowel syndrome, inflammatory bowel disease, ulcerative colitis, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, a major depressive episode (MDE), treatment resistant depression, depression in the presence of a neurodegenerative disease, cognitive impairment, Alzheimer's disease, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder, for (b) improving sleep, or for (c) improving cognition in a subject, optionally wherein for (a)-(c) the medicament is formulated for administration to a subject having an elevated TNF-α level, wherein the peptide conjugate comprises a peptide moiety attached to a conjugate moiety, wherein the peptide moiety:
a. is 5 to 15 amino acids in length; and
b. has an amino acid sequence comprising at least 5 consecutive amino acids from the amino acid sequence LSSTQAQQSX₁ (SEQ ID NO:1), where X₁ is Y, W, or F,
   optionally wherein medicament is formulated for oral administration.

336. The use of embodiment 335, wherein the peptide moiety is a peptide as described in any one of embodiments 2 to 50.

337. The use of any one of embodiments 331 to 336, wherein the medicament is formulated for administration to a subject suffering from or at risk of schizophrenia or psychosis.

338. The use of embodiment 337, wherein the medicament is formulated for administration to a subject suffering from schizophrenia.

339. The use of embodiment 337 or embodiment 338, wherein the medicament is formulated for administration to a subject exhibiting psychotic behavior.

340. The use of any one of embodiments 337 to 339, wherein administration of the medicament to the subject improves or slows progression of one or more negative symptoms of schizophrenia or psychosis.

341. The use of embodiment 340, wherein the one or more negative symptoms comprise asociality, anhedonia, alogia, affective flattening, apathy, avolition, blunted affect, anergia, apathy, depression, low mood, cognitive impairment or a combination thereof.

342. The use of embodiment 341, wherein the one or more negative symptoms comprise asociality.

343. The use of any one of embodiments 341 to 342, wherein the one or more negative symptoms comprise anhedonia.

344. The use of any one of embodiments 341 to 343, wherein the one or more negative symptoms comprise alogia.

345. The use of any one of embodiments 341 to 344, wherein the one or more negative symptoms comprise affective flattening.

346. The use of any one of embodiments 341 to 345, wherein the one or more negative symptoms comprise apathy.

347. The use of any one of embodiments 341 to 346, wherein the one or more negative symptoms comprise avolition.

348. The use of any one of embodiments 341 to 347, wherein the one or more negative symptoms comprise blunted affect.

349. The use of any one of embodiments 341 to 348, wherein the one or more negative symptoms comprise anergia.

350. The use of any one of embodiments 341 to 349, wherein the one or more negative symptoms comprise apathy.

351. The use of any one of embodiments 341 to 350, wherein the one or more negative symptoms comprise depression.

352. The use of any one of embodiments 341 to 351, wherein the one or more negative symptoms comprise low mood.

353. The use of any one of embodiments 341 to 352, wherein the one or more negative symptoms comprise cognitive impairment.

354. The use of any one of embodiments 337 to 353, wherein administration of the medicament to the subject improves or slows progression of a cognitive deficit in the subject, optionally wherein the cognitive deficit comprises a deficit in verbal working memory, spatial working memory, verbal fluency, verbal learning, or a combination thereof.

355. The use of any one of embodiments 337 to 354, wherein administration of the medicament to the subject improves or slows worsening of the subject's Positive and Negative Syndrome Scale (PANSS) negative symptom factor score.

356. The use of any one of embodiments 337 to 355, wherein administration of the medicament to the subject improves or slows worsening of the subject's Scale for the Assessment of Negative Symptoms (SANS) score.

357. The use of any one of embodiments 337 to 356, wherein administration of the medicament to the subject improves or slows worsening of the subject's Personal and Social Performance (PSP) total score.

358. The use of any one of embodiments 337 to 357, wherein administration of the medicament to the subject improves or slows worsening of the subject's PANSS total score.

359. The use of any one of embodiments 337 to 358, wherein administration of the medicament to the subject improves or slows worsening of one or more of the subject's PANSS factor scores.

360. The use of any one of embodiments 337 to 359, wherein administration of the medicament to the subject improves or slows worsening of one or more of the subject's PANSS subscale scores.

361. The use of any one of embodiments 337 to 360, wherein administration of the medicament to the subject improves or slows worsening of the subject's Clinical Global Impression Improvement (CGI-I) overall score.

362. The use of any one of embodiments 337 to 361, wherein administration of the medicament to the subject improves or slows worsening of the subject's Clinical Global Impression Improvement (CGI-I) negative symptoms rating score.

363. The use of any one of embodiments 337 to 362, wherein administration of the medicament to the subject improves or slows worsening of the subject's Clinical Global Impression Severity (CGI-S) overall score.

364. The use of any one of embodiments 337 to 363 wherein administration of the medicament to the subject improves or slows of the subject's Clinical Global Impression Severity (CGI-S) negative symptoms score.

365. The use of any one of embodiments 331 to 336, wherein the medicament is formulated for administration to a subject suffering from a neurodegenerative disease.

366. The use of embodiment 365, wherein the subject is suffering from Parkinson's disease.

367. The use of embodiment 365, wherein the subject is suffering from Alzheimer's disease.

368. The use of embodiment 365, wherein the subject is suffering from ALS.

369. The use of any one of embodiments 365 to 368, wherein the treating comprises improving or slowing progression of one or more negative symptoms of the neurodegenerative disease.

370. The use of embodiment 369, wherein the one or more negative symptoms comprise asociality, anhedonia, alogia, affective flattening, apathy, avolition, blunted affect, anergia, apathy, depression, low mood, cognitive impairment or a combination thereof.

371. The use of embodiment 370, wherein the one or more negative symptoms comprise asociality.

372. The use of embodiment 370 or embodiment 371, wherein the one or more negative symptoms comprise anhedonia.

373. The use of any one of embodiments 370 to 372, wherein the one or more negative symptoms comprise alogia.

374. The use of any one of embodiments 370 to 373, wherein the one or more negative symptoms comprise affective flattening.

375. The use of any one of embodiments 370 to 374, wherein the one or more negative symptoms comprise apathy.

376. The use of any one of embodiments 370 to 375, wherein the one or more negative symptoms comprise avolition.

377. The use of any one of embodiments 370 to 376, wherein the one or more negative symptoms comprise blunted affect.

378. The use of any one of embodiments 370 to 377, wherein the one or more negative symptoms comprise anergia.

379. The use of any one of embodiments 370 to 378, wherein the one or more negative symptoms comprise apathy.

380. The use of any one of embodiments 370 to 379, wherein the one or more negative symptoms comprise depression.

381. The use of any one of embodiments 370 to 380, wherein the one or more negative symptoms comprise low mood.

382. The use of any one of embodiments 370 to 381, wherein the one or more negative symptoms comprise cognitive impairment.

383. The use of any one of embodiments 331 to 336, wherein the medicament is formulated for administration to a subject suffering from Parkinson's disease.

384. The use of embodiment 365, wherein administration of the medicament to the subject improves or slows progression of one or more non-motor symptoms of the subject's Parkinson's disease.

385. The use of embodiment 384, wherein the one or more non-motor symptoms comprise a sensory symptom, a cognitive symptom, autonomic symptom, or a combination thereof.

386. The use of embodiment 385, wherein the one or more non-motor symptoms comprise one or more sensory symptoms.

387. The use of embodiment 386, wherein the one or more sensory symptoms comprise numbness, restlessness, pain, chest discomfort, anosmia, or a combination thereof.

388. The use of any one of embodiments 384 to 387, wherein the one or more non-motor symptoms comprise one or more cognitive symptoms.

389. The use of embodiment 388, wherein the one or more cognitive symptoms comprise mood changes, depression, anxiety, panic attacks, tiredness, confusion, slowed thinking, or a combination thereof.

390. The use of any one of embodiments 384 to 389, wherein the one or more non-motor symptoms comprise one or more autonomic symptoms.

391. The use of embodiment 390, wherein the one or more autonomic symptoms comprise hot/cold sensations, bladder problems, sweating, abdominal discomfort, constipation, sialorrhea, frequent urination and/or urgency, erectile dysfunction, or a combination thereof.

392. The use of any one of embodiments 384 to 391, wherein the one or more non-motor symptoms comprise cognition deficit and/or impairment, depression, anxiety, fatigue, apathy, or a combination thereof.

393. The use of any one of embodiments 383 to 392, wherein administration of the medicament to the subject improves or slows worsening of the subject's Non-motor Symptoms Scale (NMSS) Score.

394. The use of any one of embodiments 383 to 393, wherein administration of the medicament to the subject improves or slows worsening of the subject's MDS-UPDRS non-motor symptoms score.

395. The use of any one of embodiments 383 to 394, wherein administration of the medicament to the subject improves or slows worsening of the subject's Fatigue Severity Scale (FSS) score.

396. The use of any one of embodiments 383 to 395, wherein administration of the medicament to the subject improves or slows worsening of the subject's Beck's Depression Inventory II (BDI-II) score.

397. The use of any one of embodiments 383 to 396, wherein administration of the medicament to the subject improves or slows worsening of the subject's Beck Anxiety Inventory (BAI) score.

398. The use of any one of embodiments 383 to 397, wherein administration of the medicament to the subject improves or slows worsening of the subject's Montreal Cognitive Assessment (MoCA) score.

399. The use of any one of embodiments 383 to 398, wherein administration of the medicament to the subject improves or slows worsening of the subject's score on one or more Cogstate tests.

400. The use of any one of embodiments 383 to 399, wherein administration of the medicament to the subject improves or slows worsening of the subject's Clinical Global Impression Improvement (CGI-I) overall score.

401. The use of any one of embodiments 383 to 400, wherein administration of the medicament to the subject improves or slows worsening of the subject's Clinical Global Impression Improvement (CGI-I) non-motor symptoms score.

402. The use of any one of embodiments 383 to 401, wherein administration of the medicament to the subject improves or slows worsening of the subject's Clinical Global Impression Severity (CGI-S) overall score.

403. The use of any one of embodiments 383 to 402, wherein administration of the medicament to the subject improves or slows worsening of the subject's Clinical Global Impression Severity (CGI-S) non-motor symptoms score.

404. The use of any one of embodiments 383 to 403, wherein administration of the medicament to the subject improves or slows worsening of the subject's Parkinson's Disease Questionnaire-39 (PDQ-39) score.

405. The use of any one of embodiments 331 to 336, wherein the medicament is formulated for administration to a subject suffering from a gastrointestinal disease or disorder.

406. The use of any one of embodiments 331 to 336, wherein the medicament is formulated for administration to a subject suffering from irritable bowel syndrome.

407. The use of any one of embodiments 331 to 336, wherein the medicament is formulated for administration to a subject suffering from inflammatory bowel disease.

408. The use of any one of embodiments 331 to 336, wherein the medicament is formulated for administration to a subject suffering from ulcerative colitis.

409. The use of any one of embodiments 331 to 336, wherein the medicament is formulated for administration to a subject suffering from Crohn's disease.

410. The use of any one of embodiments 331 to 336, wherein the medicament is formulated for administration to a subject suffering from constipation.

411. The use of any one of embodiments 331 to 336, wherein the medicament is formulated for administration to a subject suffering from pain.

412. The use of any one of embodiments 331 to 336, wherein the medicament is formulated for administration to a subject suffering from visceral pain.

413. The use of any one of embodiments 331 to 336, wherein the medicament is formulated for administration to a subject suffering from rheumatoid arthritis.

414. The use of any one of embodiments 331 to 336, wherein the medicament is formulated for administration to a subject suffering from migraine.

415. The use of any one of embodiments 331 to 336, wherein the medicament is formulated for administration to a subject suffering from headache.

416. The use of any one of embodiments 331 to 336, wherein the medicament is formulated for administration to a subject suffering from substance abuse.

417. The use of any one of embodiments 331 to 336, wherein the medicament is formulated for administration to a subject suffering a substance use disorder, optionally wherein the substance use disorder is drug use disorder, polysubstance use disorder, alcohol use disorder, nicotine use disorder, or tobacco use disorder.

418. The use of any one of embodiments 331 to 336, wherein the medicament is formulated for administration to a subject suffering from drug addiction.

419. The use of embodiment 418, wherein the subject is suffering from an opioid addiction (e.g., morphine, heroin, oxycodone, or fentanyl).

420. The use of embodiment 418, wherein the subject is suffering from a cocaine addiction.

421. The use of embodiment 418, wherein the subject is suffering from a benzodiazepine addiction (e.g., diazepam, alprazolam, or clonazepam).

422. The use of any one of embodiments 331 to 336, wherein the medicament is formulated for administration to a subject suffering from a seizure disorder.

423. The use according to embodiment 422, wherein the seizure disorder is epilepsy.

424. The peptide or pharmaceutically acceptable salt thereof or peptide conjugate or pharmaceutically acceptable salt thereof for use according to embodiment 423, wherein the epilepsy is an orphan epilepsy.

425. The use of any one of embodiments 331 to 336, wherein the medicament is formulated for administration to a subject suffering from major depressive disorder.

426. The use of any one of embodiments 331 to 336, wherein the medicament is formulated for administration to a subject suffering from atypical depression.

427. The use of any one of embodiments 331 to 336, wherein the medicament is formulated for administration to a subject suffering from a major depressive episode (MDE) (e.g., atypical MDE).

428. The use of any one of embodiments 331 to 336, wherein the medicament is formulated for administration to a subject suffering from treatment resistant depression.

429. The use of any one of embodiments 331 to 336, wherein the medicament is formulated for administration to a subject suffering from depression in the presence of a neurodegenerative disease (e.g., Parkinson's disease or Alzheimer's disease).

430. The use of embodiment 429, wherein the medicament is formulated for administration to a subject suffering from depression in the presence of Parkinson's disease.

431. The use of embodiment 429, wherein the medicament is formulated for administration to a subject suffering from depression in the presence of Alzheimer's disease. 432. The use of embodiment 429, wherein the medicament is formulated for administration to a subject suffering from depression in the presence of ALS.

433. The use of any one of embodiments 331 to 336, wherein the medicament is formulated for administration to a subject suffering from cognitive impairment.

434. The use according to embodiment 433, wherein the cognitive impairment is minor cognitive impairment.

435. The use of any one of embodiments 331 to 336, wherein the medicament is formulated for administration to a subject suffering from COVID-19 related cognitive impairment and/or depression.

436. The use of any one of embodiments 331 to 336, wherein the medicament is formulated for administration to a subject suffering from Alzheimer's disease.

437. The use of any one of embodiments 331 to 336, wherein the medicament is formulated for administration to a subject suffering from ADHD.

438. The use of any one of embodiments 331 to 336, wherein the medicament is formulated for administration to a subject suffering from an autism spectrum disorder.

439. The use of any one of embodiments 331 to 336, wherein the medicament is formulated for administration to a subject suffering from a pervasive developmental disorder, which is optionally Asperger syndrome or Rett syndrome.

440. The use of any one of embodiments 331 to 336, wherein the medicament is formulated for administration to a subject suffering from atypical autism.

441. The use of any one of embodiments 331 to 336, wherein the medicament is formulated for administration to a subject suffering from multiple sclerosis.

442. The use of any one of embodiments 331 to 336, wherein the medicament is formulated for administration to a subject suffering from PTSD.

443. The use of any one of embodiments 331 to 336, wherein the medicament is formulated for administration to a subject suffering from a sleep disorder.

444. The use of any one of embodiments 331 to 336, wherein the medicament is formulated for administration to a subject suffering from insomnia.

445. The use of any one of embodiments 331 to 336, wherein the medicament is formulated for administration to a subject suffering from daytime fatigue.

446. The use of any one of embodiments 331 to 336, wherein the medicament is formulated for administration to a subject suffering from REM sleep behavior disorder.

447. The use of any one of embodiments 331 to 336, wherein the medicament is formulated for administration to a subject in need of improved sleep.

448. The use of any one of embodiments 331 to 447, wherein the subject has an elevated TNF-α level.

449. The use of any one of embodiments 331 to 336, wherein administration of the medicament improves or slows worsening of the subject's overall Patient Health Questionnaire-9 (PHQ-9) score, optionally wherein the medicament is formulated for administration to a subject who does not suffer from a condition set forth in (a) or to a healthy subject.

450. The use of embodiment 449, wherein administration of the medicament improves or slows worsening of the subject's score on one or more of questions of PHQ-9.

451. The use of embodiment 450, wherein administration of the medicament improves or slows worsening of the subject's score on one or more of PHQ-9 questions 1, 2, 3, or 6.

452. The use of any one of embodiments 331 to 336, wherein administration of the medicament improves or slows worsening of the subject's score in one or more of the CogState Groton Maze Learning Test (GMLT), the CogState Identification Test (IDN), or the CogState One Card Learning Test (OCL) optionally wherein the medicament is formulated for administration to a subject who does not suffer from a condition set forth in (a) or to a healthy subject.

453. The use of any one of embodiments 331 to 336, wherein administration of the medicament improves or slows worsening of at least one of the alpha band power or the gamma band power of the subject's electroencephalogram (EEG) optionally wherein the medicament is formulated for administration to a subject who does not suffer from a condition set forth in (a) or to a healthy subject.

454. The use of any one of embodiments 331 to 453, wherein the medicament is formulated to provide a dose of the peptide or the pharmaceutically acceptable salt thereof, or the peptide conjugate or pharmaceutically acceptable salt thereof, from about 50 mg to about 750 mg or from about 60 mg/day to about 540 mg/day.

455. The use of any one of embodiments 331 to 454, wherein the medicament is formulated to provide a dose of the peptide or pharmaceutically acceptable salt thereof, or the peptide conjugate or pharmaceutically acceptable salt thereof from once over a period of about four days to about seven times over a period of about ten days.

456. The use of any one of embodiments 331 to 454, wherein the medicament is formulated to provide a dose of about 60 mg/day of the peptide or pharmaceutically acceptable salt thereof, or the peptide conjugate or pharmaceutically acceptable salt thereof once over a period of about four days.

457. The use of any one of embodiments 331 to 454, wherein the medicament is formulated to provide a dose of about 180 mg/day of the peptide or pharmaceutically acceptable salt thereof, or the peptide conjugate or pharmaceutically acceptable salt thereof once over a period of about four days.

458. The use of any one of embodiments 331 to 454, wherein the medicament is formulated to provide a dose of about 540 mg/day of the peptide or pharmaceutically acceptable salt thereof, or the peptide conjugate or pharmaceutically acceptable salt thereof once over a period of about four days.

459. The use of any one of embodiments 331 to 454, wherein the medicament is formulated to provide a dose of about 60 mg/day of the peptide or pharmaceutically acceptable salt thereof, or the peptide conjugate or pharmaceutically acceptable salt thereof about seven times over a period of about ten days.

460. The use of any one of embodiments 331 to 454, wherein the medicament is formulated to provide a dose of about 180 mg/day of the peptide or pharmaceutically acceptable salt thereof, or the peptide conjugate or pharmaceutically acceptable salt thereof about seven times over a period of about ten days.

461. The use of any one of embodiments 331 to 454, wherein the medicament is formulated to provide a dose of about 540 mg/day of the peptide or pharmaceutically acceptable salt thereof, or the peptide conjugate or pharmaceutically acceptable salt thereof about seven times over a period of about ten days.

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. A method of (a) treating a subject suffering from or at risk of schizophrenia or psychosis, an inflammatory disease or disorder, a neurodegenerative disease such as Parkinson's disease, Alzheimer's disease or ALS, a gastrointestinal disease or disorder such as irritable bowel syndrome, inflammatory bowel disease, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, a major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder, (b) improving sleep in a subject, or (c) improving cognition in a subject, optionally wherein for (a)-(c) the subject has an elevated TNF-α level, comprising administering to the subject an effective amount of a peptide or a pharmaceutically acceptable salt thereof, wherein the peptide:
a. is 5 to 15 amino acids in length; and
b. has an amino acid sequence comprising at least 5 consecutive amino acids from the amino acid sequence LSSTQAQQSX₁ (SEQ ID NO:1), where X₁ is Y, W, or F.

2. The method of claim 1, wherein the amino acid sequence of the peptide or pharmaceutically acceptable salt thereof is LSSTQAQQSY (SEQ ID NO:2).

3. The method of claim 1 or claim 2, wherein the peptide or pharmaceutically acceptable salt thereof is administered orally.

4. A method of (a) treating a subject suffering from or at risk of schizophrenia or psychosis, an inflammatory disease or disorder, a neurodegenerative disease such as Parkinson's disease, Alzheimer's disease, or ALS, a gastrointestinal disease or disorder such as irritable bowel syndrome, inflammatory bowel disease, Crohn's disease, or constipation, pain (e.g., visceral pain), rheumatoid arthritis, migraine, headache, substance abuse (e.g., substance use disorder such as drug use disorder, polysubstance use disorder, alcohol use disorder, nicotine use disorder, or tobacco use disorder), drug addiction, a seizure disorder, major depressive disorder, atypical depression, a major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, a sleep disorder such as insomnia, daytime fatigue, or REM sleep behavior disorder, (b) improving sleep in a subject, or (c) improving cognition in a subject, optionally wherein for (a)-(c) the subject has an elevated TNF-α level, comprising administering to the subject an effective amount of a peptide conjugate or a pharmaceutically acceptable salt thereof, wherein the peptide conjugate comprises a peptide moiety attached to a conjugate moiety, wherein the peptide moiety:
a. is 5 to 15 amino acids in length; and
b. has an amino acid sequence comprising at least 5 consecutive amino acids from the amino acid sequence LSSTQAQQSX₁ (SEQ ID NO:1), where X₁ is Y, W, or F.

5. The method of claim 4, wherein the peptide conjugate or pharmaceutically acceptable salt thereof is administered orally.

6. The method of any one of claims 1 to 5, wherein the subject is suffering from or at risk of schizophrenia or psychosis.

7. The method of claim 6, wherein the subject is suffering from schizophrenia.

8. The method of any one of claims 6 to 7, wherein the subject exhibits psychotic behavior.

9. The method of any one of claims 6 to 8, wherein the treating comprises improving or slowing progression of one or more negative symptoms of schizophrenia or psychosis.

10. The method of claim 9, wherein the one or more negative symptoms comprise asociality, anhedonia, alogia, affective flattening, apathy, avolition, blunted affect, anergia, apathy, depression, low mood, cognitive impairment, or a combination thereof.

11. The method of any one of claims 6 to 10, wherein the treating comprises improving or slowing progression of a cognitive deficit in the subject, optionally wherein the cognitive deficit comprises a deficit in verbal working memory, spatial working memory, verbal fluency, verbal learning, or a combination thereof.

12. The method of any one of claims 1 to 5, wherein the subject is suffering from a neurodegenerative disease, which is optionally Parkinson's disease, Alzheimer's disease, or ALS.

13. The method of claim 12, wherein the treating comprises improving or slowing progression of one or more negative symptoms of the neurodegenerative disease.

14. The method of claim 13, wherein the one or more negative symptoms comprise asociality, anhedonia, alogia, affective flattening, apathy, avolition, blunted affect, anergia, apathy, depression, low mood, cognitive impairment or a combination thereof.

15. The method of any one of claims 1 to 5 and 12 to 14, wherein the subject is suffering from Parkinson's disease.

16. The method of claim 15, wherein the treating comprises improving or slowing progression of one or more non-motor symptoms of the subject's Parkinson's disease.

17. The method of claim 16, wherein the one or more non-motor symptoms comprise a sensory symptom, a cognitive symptom, autonomic symptom, or a combination thereof.

18. The method of claim 17, wherein the one or more sensory symptoms comprise numbness, restlessness, pain, chest discomfort, anosmia, or a combination thereof; the one or more cognitive symptoms comprise mood changes, depression, anxiety, panic attacks, tiredness, confusion, slowed thinking, or a combination thereof; and the one or more autonomic symptoms comprise hot/cold sensations, bladder problems, sweating, abdominal discomfort, constipation, sialorrhea, frequent urination and/or urgency, erectile dysfunction, or a combination thereof.

19. The method of any one of claims 16 to 18, wherein the one or more non-motor symptoms comprise cognition deficit and/or impairment, depression, anxiety, fatigue, apathy, or a combination thereof.

20. The method of any one of claims 1 to 5, wherein the subject is suffering from a gastrointestinal disease or disorder.

21. The method of any one of claims 1 to 5, wherein the subject is suffering from irritable bowel syndrome.

22. The method of any one of claims 1 to 5, wherein the subject is suffering from inflammatory bowel disease.

23. The method of any one of claims 1 to 5, wherein the subject is suffering from Crohn's disease.

24. The method of any one of claims 1 to 5, wherein the subject is suffering from constipation.

25. The method of any one of claims 1 to 5, wherein the subject is suffering from pain.

26. The method of any one of claims 1 to 5, wherein the subject is suffering from visceral pain.

27. The method of any one of claims 1 to 5, wherein the subject is suffering from rheumatoid arthritis.

28. The method of any one of claims 1 to 5, wherein the subject is suffering from migraine.

29. The method of any one of claims 1 to 5, wherein the subject is suffering from headache.

30. The method of any one of claims 1 to 5, wherein the subject is suffering from substance abuse.

31. The method of any one of claims 1 to 5, wherein the subject is suffering from a substance use disorder, optionally wherein the substance use disorder is drug use disorder, polysubstance use disorder, alcohol use disorder, nicotine use disorder, or tobacco use disorder.

32. The method of any one of claims 1 to 5, wherein the subject is suffering from drug addiction, optionally wherein the drug addiction is opioid addiction, cocaine addiction, or benzodiazepine addiction.

33. The method of any one of claims 1 to 5, wherein the subject is suffering from a seizure disorder.

34. The method of claim 33, wherein the seizure disorder is epilepsy.

35. The method of claim 33, wherein the epilepsy is an orphan epilepsy.

36. The method of any one of claims 1 to 5, wherein the subject is suffering from major depressive disorder.

37. The method of any one of claims 1 to 5, wherein the subject is suffering from atypical depression.

38. The method of any one of claims 1 to 5, wherein the subject is suffering from a major depressive episode (MDE) (e.g., atypical MDE).

39. The method of any one of claims 1 to 5, wherein the subject is suffering from treatment resistant depression.

40. The method of any one of claims 1 to 5, wherein the subject is suffering from depression in the presence of a neurodegenerative disease.

41. The method of any one of claims 1 to 5, wherein the subject is suffering from cognitive impairment.

42. The method of claim 41, wherein the cognitive impairment is minor cognitive impairment.

43. The method of any one of claims 1 to 5, wherein the subject is suffering from COVID-19 related cognitive impairment and/or depression.

44. The method of any one of claims 1 to 5, wherein the subject is suffering from Alzheimer's disease.

45. The method of any one of claims 1 to 5, wherein the subject is suffering from ADHD.

46. The method of any one of claims 1 to 5, wherein the subject is suffering from an autism spectrum disorder.

47. The method of any one of claims 1 to 5, wherein the subject is suffering from a pervasive developmental disorder, which is optionally Asperger syndrome or Rett syndrome.

48. The method of any one of claims 1 to 5, wherein the subject is suffering from atypical autism.

49. The method of any one of claims 1 to 5, wherein the subject is suffering from multiple sclerosis.

50. The method of any one of claims 1 to 5, wherein the subject is suffering from PTSD.

51. The method of any one of claims 1 to 5, wherein the subject is suffering from a sleep disorder.

52. The method of any one of claims 1 to 5, wherein the subject is suffering from insomnia.

53. The method of any one of claims 1 to 5, wherein the subject is suffering from daytime fatigue.

54. The method of any one of claims 1 to 5, wherein the subject is suffering from REM sleep behavior disorder.

55. The method of any one of claims 1 to 5, wherein the treating comprises improving sleep in a subject.

56. The method of any one of claims 1 to 55, wherein the peptide or a pharmaceutically acceptable salt thereof or peptide conjugate or a pharmaceutically acceptable salt thereof is administered at a dose from about 50 mg/day to 750 mg/day or about 60 mg/day to about 540 mg/day.

57. The method of claim 56, wherein the dose is 60 mg/day, 180 mg/day, or 540 mg/day.
